(19) United States
(12) Patent Application Publication
(10) Pub. No.: US 2025/0170082 A1
(43) Pub. Date: May 29, 2025

(54) HPDL INHIBITORS AND USES THEREOF

(71) Applicant: NEW YORK UNIVERSITY, New York, NY (US)

(72) Inventors: Michael PACOLD, New York, NY (US); Robert BANH, New York, NY (US); Quentin SPILLIER, Coutisse (BE)

(73) Assignee: NEW YORK UNIVERSITY, New York, NY (US)

(21) Appl. No.: 18/840,970

(22) PCT Filed: Mar. 3, 2023
(86) PCT No.: PCT/US2023/014468

§ 371 Date: Aug. 23, 2024

Related U.S. Application Data
(60) Provisional application No. 63/316,196, filed on Mar. 3, 2022.

(51) Int. Cl. A61K 31/192; A61K 31/137; A61K 31/195; A61K 31/47; A61K 45/06; A61P 35/00

(52) U.S. Cl. CPC A61K 31/192; A61K 31/137; A61K 31/195; A61K 31/47; A61K 45/06; A61P 35/00

(57) ABSTRACT

Various methods of treating cancer, autoimmune disease, or any disease involving cellular proliferation are presented herein comprising administering an effective amount of an HPDL inhibitor to the subject. The HPDL inhibitors used herein are ibuprofen, dopamine, or their derivatives. Also presented herein are methods of determining effectiveness of a treatment for cancer, autoimmune disease, or any disease involving cellular proliferation in a subject, comprising administering ibuprofen, dopamine, or their derivatives. Further presented herein are methods of predicting prognosis of cancer in a subject having cancer, autoimmune disease, or any disease involving cellular proliferation, wherein if the subject is determined to have poor prognosis, the method further comprising administering to the subject a single-agent chemotherapy, supportive care, and/or a compound that is ibuprofen, dopamine, or their derivatives.

### CROSS-REFERENCE TO RELATED APPLICATIONS

This application claims priority to U.S. Provisional Application No. 63/316,196, filed Mar. 3, 2022, the disclosure of which is herein incorporated by reference in its entirety.

### STATEMENT REGARDING FEDERALLY SPONSORED RESEARCH OR DEVELOPMENT

This invention was made with government support under K22 CA212059 awarded by the National Institutes of Health. The government has certain rights in the invention.

### FIELD OF THE INVENTION

The invention provides methods of treating cancer, autoimmune disease, or any disease involving cellular growth or proliferation by administering an effective amount of a 4-hydroxyphenylpyruvate dioxygenase-like (HPDL) inhibitor. The HPDL inhibitors comprise ibuprofen, dopamine, and their derivatives.

### BACKGROUND

Coenzyme Q (CoQ), or ubiquinone, is a ubiquitous cofactor found in all eukaryotes and in most bacteria. Coenzyme Q consists of a 1,4 benzoquinone headgroup that can be reversibly oxidized or reduced, attached to an isoprenoid tail of varying lengths (1). In humans, this tail contains 10 isoprene units. For this reason, human Coenzyme Q is called Coenzyme Q10 (CoQ10). In mammalian cells, CoQ10 is found in most biological membranes, where it is thought to function as an antioxidant (2). In the mitochondrion, CoQ10 accepts electrons from respiratory complexes I and II and transfers them to complex III. CoQ10 is critical for the electron transport chain and for mitochondrial function because it is the only lipid-soluble, endogenously synthesized single-electron carrier in the cell, which allows it to transfer electrons to and from the iron-sulfur clusters in the enzymes of the electron transport chain (3).

Cells require CoQ10 for mitochondrial function and must maintain an adequate supply of this coenzyme for growth and survival. It has long been known that CoQ10 can be absorbed in limited quantities from the diet and that it likely is synthesized at multiple sites in the cell (4-6). The CoQ10 tail synthesis pathway, which is a branch of the mevalonate pathway, has been studied for decades (7). Because both cholesterol and the CoQ10 tail are derived from mevalonate, CoQ10 supplementation is recommended for management of statin-induced muscle symptoms (SAMS), but the results of CoQ10 supplementation trials for treatment of SAMS are equivocal (8), in part due to an incomplete understanding of CoQ10 absorption and pharmacokinetics. In contrast to the CoQ10 tail synthesis reactions, the biosynthetic reactions of the CoQ10 headgroup are not fully characterized in humans (9). It is known that tyrosine is the precursor for the CoQ10 quinone headgroup, and that tyrosine is catabolized to 4-hydroxyphenylpyruvate (4-HPPA), which is in turn converted by a series of unknown reactions to 4-hydroxybenzoate (4-HB). 4-HB is conjugated by the COQ2 enzyme to the polyprenyl tail that anchors CoQ10 in the membrane (10) (FIG. 1A). There has been recent progress in identifying some of the CoQ10 headgroup synthesis genes in yeast (11), but the enzymes of CoQ10 headgroup synthesis in humans remain unknown. The discovery of new human CoQ10 biosynthesis genes and intermediates is important because loss of known CoQ10 synthesis genes results in childhood neurodegenerative diseases that often present with encephalopathy and seizures (12-15). In addition, CoQ10 is important for mitochondrial electron transport chain (ETC) activity, which is necessary for cell growth (14,15). Revealing the mechanisms that proliferating cells use to maintain CoQ10 biosynthesis and ETC activity will improve our understanding of the metabolic requirements supporting deranged cell proliferation in cancer and autoimmune diseases and enable the development of CoQ10 synthesis inhibitors to control these diseases.

It was discovered that 4-hydroxymandelate (4-HMA) is a novel intermediate in the human CoQ10 headgroup biosynthesis pathway, and 4-hydroxyphenylpyruvate dioxygenase-like (HPDL) (FIG. 1A) is identified as the enzyme that makes it (16). HPDL is a previously unannotated mitochondrial iron-dependent dioxygenase that catalyzes the oxidative decarboxylation of 4-HPPA to 4-HMA, which is the first irreversible step in the human CoQ10 headgroup synthesis pathway. It was found that HPDL deletion decreases the incorporation of labeled tyrosine into CoQ10 and reduces the formation of orthotopically implanted pancreatic ductal adenocarcinoma (PDAC) tumors. This defect is restored by addback of wild-type HPDL. In contrast, addback of catalytically inactive HPDL fails to restore the growth of HPDL-null tumors. Multiple missense and nonsense HPDL mutations were shown to segregate with a childhood neurodegenerative disorder presenting with spasticity and encephalopathy, and an inherited form of cerebral palsy (17-20). In addition, HPDL overexpression accelerates the growth of PDAC cells (21) and breast cancers. These studies did not elucidate the reaction catalyzed by HPDL or the mechanisms for these phenotypes. The work showing that HPDL catalyzes a critical step in CoQ10 headgroup synthesis explains these phenotypes and suggests that HPDL inhibition could decrease the growth of cells in benign and malignant conditions.

As such, there is an unmet need for novel therapeutics based on HPDL inhibition for treatment of various diseases and conditions, including HPDL-dependent tumors, cancers (e.g., pancreatic and breast cancers), autoimmune diseases, and any diseases involving unnecessary or uncontrolled cellular proliferation or mitochondrial activation.

### SUMMARY OF THE INVENTION

The present invention addresses the unmet need specified in the Background Section, above, and other needs by providing HPDL inhibitors for use in such treatment.

In one aspect, provided herein is a method of treating cancer, autoimmune disease, or any disease involving cellular proliferation in a subject in need thereof, comprising the following steps:
a. determining expression level of 4-hydroxyphenylpyruvate dioxygenase-like (HPDL) in a sample obtained from the subject;
b. comparing the HPDL expression level determined in step (a) with a control level of HPDL expression; and
c. administering an effective amount of an HPDL inhibitor to the subject exhibiting a higher level of HPDL expression as compared to the control level, wherein the HPDL inhibitor is a compound that is ibuprofen or an ibuprofen derivative having the structure of Formula (I), dopamine or a dopamine derivative having the structure of Formula (II), flurbiprofen, ellagic acid, methyldopa, disulfiram, idelalisib, eltrombopag olamine, crystal violet, verteporfin, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations thereof,

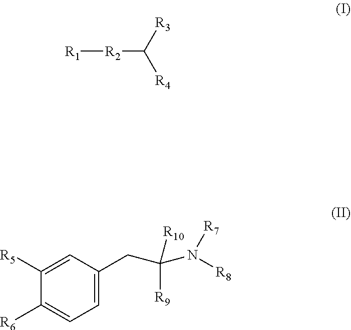

wherein
R₁ is selected from C₁₋₁₂ alkyl, —NH₂, and phenyl optionally substituted by one or more of halogen, —OH, and combinations thereof,
R₂ is selected from C₁₋₆ alkyl and phenyl optionally substituted by one or more of halogen, —OH, and combinations thereof, and
R₃ and R₄ are independently selected from hydrogen, C₁₋₆ alkyl, —NH₂, —CO₂H, phenyl optionally substituted by one or more of halogen, —OH, and combinations thereof,
R₅ and R₆ are independently selected from —OH and —CO₂H;
R₇ and R₈ are independently selected from hydrogen and —NH₂, and
R₉ is selected from hydrogen and —CO₂H, R₁₀ is selected from hydrogen and C₁₋₆ alkyl,
or a pharmaceutically acceptable salt thereof.

In another aspect, provided herein is a method of determining effectiveness of a treatment for cancer, autoimmune disease, or any disease involving cellular proliferation in a subject, comprising the following steps:
a. determining expression level of 4-hydroxyphenylpyruvate dioxygenase-like (HPDL) in a first sample obtained from the subject before the subject receives a treatment for cancer, autoimmune disease, or any disease involving cellular proliferation in a subject comprising administering a compound that is ibuprofen or an ibuprofen derivative having the structure of Formula (I), dopamine or a dopamine derivative having the structure of Formula (II), flurbiprofen, ellagic acid, methyldopa, disulfiram, idelalisib, eltrombopag olamine, crystal violet, verteporfin, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations thereof,

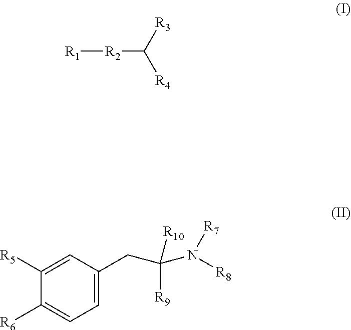

wherein
R₁ is selected from C₁₋₁₂ alkyl, —NH₂, and phenyl optionally substituted by one or more of halogen, —OH, and combinations thereof,
R₂ is selected from C₁₋₆ alkyl and phenyl optionally substituted by one or more of halogen, —OH, and combinations thereof, and
R₃ and R₄ are independently selected from hydrogen, C₁₋₆ alkyl, —NH₂, —CO₂H, phenyl optionally substituted by one or more of halogen, —OH, and combinations thereof,
R₅ and R₆ are independently selected from —OH and —CO₂H;
R₇ and R₈ are independently selected from hydrogen and —NH₂, and
R₉ is selected from hydrogen and —CO₂H,
R₁₀ is selected from hydrogen and methyl,
or a pharmaceutically acceptable salt thereof;
b. determining expression level of HPDL in a second sample obtained from the subject after the subject has received the treatment for cancer, autoimmune disease, or any disease involving cellular proliferation;
c. comparing the HPDL expression levels determined in the first sample and the second sample; and
d. determining that (i) the treatment is effective if the HPDL expression level in the second sample is lower than HPDL expression level in the first sample, or (ii) the treatment is not effective if the HPDL expression level in the second sample is not lower than the HPDL expression level in the first sample.

In an additional aspect, provided herein is a method of predicting prognosis of cancer in a subject having cancer, autoimmune disease, or any disease involving cellular proliferation, comprising the following steps:
a. determining expression level of 4-hydroxyphenylpyruvate dioxygenase-like (HPDL) protein in a sample obtained from the subject;
b. comparing the HPDL expression level determined in step (a) with a control level of HPDL expression; and
c. determining the subject (i) as having poor prognosis if the HPDL expression level is higher than the control level, or (ii) as having good prognosis if the HPDL expression level is lower than or equal to the control level, wherein if the subject is determined to have poor prognosis, the method further comprising administering to the subject a single-agent chemotherapy, supportive care, and/or a compound that is ibuprofen or an ibuprofen derivative having the structure of Formula (I), dopamine or a dopamine derivative having the structure of Formula (II), flurbiprofen, ellagic acid, methyldopa, disulfiram, idelalisib, eltrombopag olamine, crystal violet, verteporfin, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations thereof,

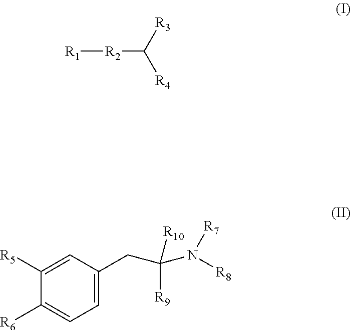

wherein
R₁ is selected from C₁₋₁₂ alkyl, —NH₂, and phenyl optionally substituted by one or more of halogen, —OH, and combinations thereof,
R₂ is selected from C₁₋₆ alkyl and phenyl optionally substituted by one or more of halogen, —OH, and combinations thereof, and
R₃ and R₄ are independently selected from hydrogen, C₁₋₆ alkyl, —NH₂, —CO₂H, phenyl optionally substituted by one or more of halogen, —OH, and combinations thereof,
R₅ and R₆ are independently selected from —OH and —CO₂H;
R₇ and R₈ are independently selected from hydrogen and —NH₂, and
R₉ is selected from hydrogen and —CO₂H,
R₁₀ is selected from hydrogen and methyl,
or a pharmaceutically acceptable salt thereof.

### BRIEF DESCRIPTION OF THE DRAWINGS

FIG. 1A shows incorporation of tyrosine into the CoQ10 headgroup via HPDL. 4-HMA. 4-hydroxyphenylpyruvate (4-HPPA) is a canonical catabolite of tyrosine. It is discovered that the enzyme hydroxy-phenylpyruvate dioxygenase-like (HPDL) converts 4-HPPA to 4-hydroxymandelate (4-HMA), which is converted to 4-hydroxybenzoate (4-HB), the immediate precursor of the CoQ10 headgroup.

FIG. 1B shows fractions from HPDL purification run on a Coomassie stained gel of high purity after Talon metal affinity and cation exchange. It is demonstrated that His-tagged HPDL can be produced and purify using expiSF9 expression system.

FIG. 2 shows GC-MS tracking of 4HMA allows to follow HPDL activity after purification. Panel (A) is a schematic representation of HPDL catalytic reaction, Panel (B) shows GC-MS detection of 4HMA produced following enzymatic assay on purified enzyme, and Panel (C) demonstrates GC-MC kinetic characterization of HPDL activity.

FIG. 3 shows an overview of HPDL thermal stabilization and/or destabilization from a representative panel of compounds after evaluation at 10 μM (blue <2° C. shift, green >2° C. shift). It is indicated that SelleckChem FDA approved library contains potential HPDL binders.

FIG. 4 shows the binders act as HPDL inhibitors. Panel (A) shows dose-response curve of representative binders on HPDL. The four curves in Panel (A) represent dose-response curves for, from left to right, ibuprofen, dopamine, montelukast, and methyldopa, respectively. At 50% activity level, the compound amount is least for ibuprofen and second least for dopamine. Panel (B) shows chemical structures and IC50 values obtained from these molecules. From left to right in Panel (B), structures and the corresponding IC50 are shown for montelukast, dopamine, methyldopa, and ibuprofen.

FIG. 5 demonstrates 3D growth assays in MIAPACA2 and PATU-8902 cells after 3 days of treatment with ibuprofen. It is shown that ibuprofen selectively decreases cell growth of HPDL-dependent cell lines in 3D.

FIG. 6 shows 3D growth assays conducted in MIAPACA2 and PATU-8902 cells after 3 days of treatment with ibuprofen, 4HMA, and both molecules. ***P<0.005. It is shown that 4HMA rescues ibuprofen-induced decreases cell growth in 3D.

FIG. 7 shows thermal shift curves obtained with a Sypro orange assay of ibuprofen and aspirin on purified HPDL. It is shown that ibuprofen acts as a HPDL binder, while aspirin does not act HPDL binder.

FIG. 8 shows 3D growth assays conducted in MIAPACA2 cells after 3 days of treatment with ibuprofen and aspirin. ***P<0.005. It is shown that aspirin does not affect cell growth of HPDL-dependent cell lines and ibuprofen affects cell growth of HPDL-dependent cell lines in 3D.

FIG. 9 shows 3D growth assays conducted in MIAPACA2 cells after 3 days of treatment with ibuprofen. ***P<0.005. It is shown that HPDL deletion reduces sensitivity to ibuprofen treatment in 3D.

FIG. 10 shows ibuprofen affects the growth of HPDL-dependent PDAC orthotopic xenografts. Panel (A) is a schematic overview of the in vivo experiment. Ibuprofen and 4HMA were supplemented into mouse drinking water. Panel (B) shows orthotopic pancreatic tumor weight from MIAPACA2 and PATU8902 xenografts after sacrificing the animals.

FIG. 11 shows survival plots after daily oral gavage of ibuprofen, 4HMA and both molecules. The treatment was started on 3 different aged pup groups. It is shown that 4HMA supplementation rescues ibuprofen-induced lethality in pups.

FIG. 12 shows docking simulation of ibuprofen and HPPA in HPDL active site with three residues interacting with the compound (H258, H163 and Q324). Alphafold predicted model is used for HPDL. It is shown that ibuprofen and HPPA show an identical interaction pattern with HPDL.

### DETAILED DESCRIPTION OF THE INVENTION

Some aspects of the present application are based on the discovery that HPDL catalyzes a critical step in the synthesis of the headgroup of Coenzyme Q10 (CoQ10). HPDL makes R-4-hydroxymandelate ((R)-4-HMA), which is a long sought intermediate in the synthesis of the CoQ10 headgroup. R-4-HMA is converted to 4-hydroxybenzoic acid (4-HB). 4-HMA is chiral, with both (R) and (S)-enantiomers theoretically possible, and HPDL appears to produce exclusively the R-enantiomer of this molecule. The data here also show that 4-HMA is readily taken up by cells and incorporated into CoQ10.

Because the pharmacokinetics of CoQ10 are not well understood and it is not clear how much orally ingested CoQ10 is actually absorbed, and because 4-HMA is soluble, the natural product of HPDL, and is taken up by cells, supplementation with 4-HMA, 4-HB, or other intermediates (e.g., 4-hydroxybenzoylformate (4-HBF), 4-hydroxybenzaldehyde (4-HBz)) should treat patients with CP or neurodevelopmental disease induced by HPDL mutations. If CoQ10 were readily absorbed and entered the brain, it could treat this disease as well.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values+20% of the recited value, e.g. “about 90%” may refer to the range of values from 71% to 99%.

As used herein, the terms “hermetically sealed” and “closed system” refer to a configuration of a system or an apparatus in which gasses are not permitted to flow but for flow through specifically designated inlet and/or outlet ports. An inlet port can be hermetically sealed by virtue of being mechanically connected with gas-impermeable materials to a controlled gas flow or by being hermetically closed (e.g. by a gas-impermeable closed valve). An outlet port can be hermetically sealed by virtue of being hermetically closed or by virtue of gas flow in only a single direction, i.e. out of the closed system.

As used herein, the term “alkyl” is given its ordinary meaning in the art and can include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 1-20 carbon atoms in its backbone (e.g., C₁₋₂₀ for straight chain, C₂₋₂₀ for branched chain), and alternatively, about 1-10 carbon atoms, or about 1 to 6 carbon atoms. In some embodiments, a cycloalkyl ring has from about 3-10 carbon atoms in their ring structure where such rings are monocyclic or bicyclic, and alternatively about 5, 6 or 7 carbons in the ring structure. In some embodiments, a cycloalkyl group is a cyclopropyl, a cyclobutyl, a cyclopentyl, or a cyclohexyl group. In some embodiments, an alkyl group can be a lower alkyl group, wherein a lower alkyl group comprises 1-4 carbon atoms (e.g., C₁₋₄ for straight chain lower alkyls). When used in the context of a divalent alkyl group, it is to be understood that “alkyl” refers to an alkylene group.

As used herein, the term “alkenyl” refers to an alkyl group, as defined herein, including straight-chain alkenyl groups, branched-chain alkenyl groups, and cycloalkenyl groups having one or more double bonds. In certain embodiments, a straight chain or branched chain alkenyl has about 1-20 carbon atoms in its backbone (e.g., C₂₋₂₀ for straight chain, C₃₋₂₀ for branched chain), and alternatively, about 2-10 carbon atoms, or about 2 to 6 carbon atoms. In some embodiments, an alkenyl group has 1, 2, 3, 4, 5, or 6 double bonds. In some embodiments, a cycloalkenyl ring has from about 3-10 carbon atoms in the ring structure where such rings are monocyclic or bicyclic, and alternatively about 5, 6 or 7 carbons in the ring structure, and 1, 2, or 3 double bonds. In some embodiments, a cycloalkenyl group is a cyclopropenyl, a cyclobutenyl, a cyclobutadienyl, a cyclopentenyl, a cyclopentadienyl, a cyclohexenyl, or a cyclohexadienyl group. In some embodiments, an alkenyl group can be a lower alkenyl group, wherein a lower alkenyl group comprises 2-4 carbon atoms (e.g., C₂₋₄ for straight chain lower alkenyls). In one embodiment, a cycloalkenyl group has six carbon atoms and one double bond.

As used herein, the term “alkynyl” refers to an alkyl group, as defined herein, including straight-chain alkynyl groups, branched-chain alkynyl groups, and cycloalkynyl groups having one or more triple bonds. In certain embodiments, a straight chain or branched chain alkynyl has about 2-20 carbon atoms in its backbone (e.g., C₂₋₂₀ for straight chain, C₃₋₂₀ for branched chain), and alternatively, about 2-10 carbon atoms, or about 2 to 6 carbon atoms. In some embodiments, an alkynyl group has 1, 2, 3, 4, 5, or 6 triple bonds. In some embodiments, a cycloalkynyl ring has from about 6-12 carbon atoms in the ring structure where such rings are monocyclic or bicyclic, and alternatively about 8, 9, or 10 carbons in the ring structure, and 1, 2, or 3 triple bonds. In some embodiments, an alkynyl group can be a lower alkynyl group, wherein a lower alkynyl group comprises 2-4 carbon atoms (e.g., C₂₋₄ for straight chain lower alkynyls).

The term “heteroalkyl” is given its ordinary meaning in the art and refers to alkyl groups as described herein in which one or more carbon atoms is replaced with a heteroatom (e.g., oxygen, nitrogen, sulfur, and the like). In some embodiments, a heteroalkyl group can have one or more of methylene groups replaced with —O—, —S—, or —NH—, in which the hydrogen of —NH— is optionally substituted. Examples of heteroalkyl groups include, but are not limited to, alkoxy, poly(ethylene glycol)-, alkyl-substituted amino, tetrahydrofuranyl, piperidinyl, morpholinyl, etc.

The term “haloalkyl” is given its ordinary meaning in the art and refers to alkyl groups as described herein in which one or more hydrogen atoms is replaced by a halogen atom, i.e., F, Cl, Br, or I. In some embodiments a haloalkyl group can be a perfluoroalkyl group, i.e., a group where all hydrogen atoms are replaced with fluoride atoms. In some embodiments a haloalkyl group can be a halomethyl group, i.e., a C₁ group with 1, 2, or 3 halogen atoms, e.g., —CF₃, —CF₂H, —CH₂F, —CH₂Cl, —CH₂Br; —CH₂I. In some embodiments a haloalkyl group can be, e.g., —CF₃, —CF₂H, —CH₂F, —CH₂Cl, —CH₂Br; —CH₂I, —CH₂CF₃, CH₂CH₂F, —CH₂CH₂Br, —CH₂CH₂Cl, —CH₂CH₂I, etc.

The term “haloalkoxy” is given its ordinary meaning in the art and refers to alkoxy groups as described herein, i.e. alkyl groups bonded to an oxygen atom, in which one or more hydrogen atoms is replaced by a halogen atom, i.e., F, Cl, Br, or T. In some embodiments a haloalkoxy group can be a perfluoroalkoxy group, i.e., a group where all hydrogen atoms are replaced with fluoride atoms. In some embodiments a haloalkoxy group can be, e.g., —OCF₃, —OCF₂H, —OCH₂F, —OCH₂Cl, —OCH₂Br; —OCH₂I, —OCH₂CF₃, —OCH₂CH₂F, —OCH₂CH₂Br, —OCH₂CH₂Cl, —OCH₂CH₂I, etc.

The term “aryl” used alone or as part of a larger moiety as in “aralkyl,” “aralkoxy,” “aryloxy” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” can be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which can bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The term “aralkyl” refers to alkyl groups as described herein in which one or more hydrogen atoms is substituted by aryl groups, where the radical or point of attachment is on the alkyl group. The alkyl part of an aralkyl group is optionally substituted as described in the term “alkyl” above. The aryl part of the aralkyl group is optionally substituted as described in ther term “alkyl” above.

The term “alkylaryl” referes to aryl groups as described herein in which one or more hydrogen atoms is substituted by alkyl groups, where the radical or point of attachment is on the aryl group. The aryl part of the alkylaryl group is optionally substituted as described in the term “aryl” above. The alkyl part of an alkylaryl group is optionally substituted as described in the term “alkyl” above.

The terms “heteroaryl” and “heteroar-,” used alone of as part of a larger moiety, e.g., “heteroaralkyl,” or “heteroaralkoxy,” refer to groups having 5 to 10 ring atoms (i.e., monocyclic or bicyclic), in some embodiments 5, 6, 9, or 10 ring atoms. In some embodiments, such rings have 6, 10, or 14 T electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, pyridine, quinoline, isoquinoline, quinolizine, pyrido[1,2-a]pyrazine, 1,8-naphthyridine, purine, chromene, indole, phenanthrene, benzo[H]quinoline, anthraquinone, and phenanthrol[1,2-b]furan groups. In some embodiments, a heteroaryl is a heterobiaryl group, such as bipyridyl and the like. The terms “heteroaryl” and “heteroar-,” as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group can be monocyclic, bicyclic, tricyclic, tetracyclic, and/or otherwise polycyclic. The term “heteroaryl” can be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is saturated, partially unsaturated, or aromatic, and having, in addition to carbon atoms, one or more, e.g., one to four, heteroatoms, as defined above. As used herein, the term “heterocycle” encompasses heteroaryl groups, as defined above. In one embodiment, a heterocycle can be a saturated, partially unsaturated, or aromatic, 5-7 membered monocyclic moiety comprising from 1 to 3 nitrogen atoms, e.g., a pyrrole, an imidazole, a pyrazole, a pyrazole, a triazole, a piperidine, a piperazine, a pyridazine, a pyridine, 2H-pyridine, a pyridone, a pyrimidine, or a pyrazine, including monovalent or divalent radicals thereof. When used in reference to a ring atom of a heterocycle, the term “nitrogen” includes a substituted nitrogen. In one embodiment, a heterocycle can be a saturated, partially unsaturated, or aromatic, 5-7 membered monocyclic moiety comprising from 1 to 3 oxygen atoms, e.g., a tetrahydrofuran (i.e., oxolane), a furan, a dihydrofuran, a dioxolane, a tetrahydropyran (i.e., oxane), a pyran, a dihydropyran, a dioxane, a dioxine, a trioxane, an oxepane, or an oxepine, including monovalent or divalent radicals thereof. In one embodiment, a heterocycle can be thiophene, oxazole, thiazole, or morpholine, including monovalent or divalent radicals thereof.

A heterocyclic ring can be attached, e.g., to its pendant group, at any heteroatom or carbon atom that results in a stable structure. Examples of such saturated or partially unsaturated heterocyclic radicals include, without limitation, tetrahydropyranyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as phenyl, indolinyl, 3H-indolyl, chromanyl, phenanthridinyl, or tetrahydroquinolinyl. A heterocyclyl group can be monocyclic, bicyclic, tricyclic, tetracyclic, and/or otherwise polycyclic. The term “heterocyclylalkyl” refers to an alkyl group substituted by a heterocyclyl.

The term “heteroatom” means one or more of oxygen, sulfur, nitrogen, phosphorus, or silicon, including any oxidized form of nitrogen, sulfur, phosphorus, or silicon; the quaternized form of any basic nitrogen. In some embodiments, a heteroatom can be a substitutable nitrogen of a heterocyclic ring.

The term “unsaturated,” as used herein, means that a moiety has one or more units of unsaturation.

The term “halogen” means F, Cl, Br, or I atom, and/or its radical or substituent, namely —F, —Cl, —Br, or —I.

As described herein, in certain embodiments, certain compounds of the disclosure can be indicated to comprise “optionally substituted” moieties. When indicated, in general, the term “substituted,” whether preceded by the term “optionally” or not, means that one or more hydrogens of the designated moiety are replaced with a suitable substituent. Unless otherwise indicated, an “optionally substituted” group can have a suitable substituent at each substitutable position of the group, and when more than one position in any given structure can be substituted with more than one substituent selected from a specified group, the substituent can be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

As used herein, a substituent, e.g., —B, can be represented as

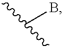

where [symbol] denotes a point of attachment.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the disclosure.

The present application also includes pharmaceutically acceptable salts of the compounds described herein. The “pharmaceutically acceptable salts” include a subset of the “salts” described above which are conventional non-toxic salts of the parent compound formed, for example, from non-toxic inorganic or organic acids. Lists of suitable salts are found in Remington's Pharmaceutical Sciences, 17th ed., Mack Publishing Company, Easton, Pa., 1985, p. 1418 and Berge, S M et al, Journal of Pharmaceutical Science, 1977, 66, 1, 1-19. By way of an example, in an embodiment of the disclosure pharmaceutically acceptable salts can comprise a suitable anion selected from F⁻, Cl⁻, Br⁻, I⁻, OH⁻, —BF₄, CF₃SO₃⁻, monobasic sulfate, dibasic sulfate, monobasic phosphate, dibasic phosphate, or tribasic phosphate, NO₃⁻, PF⁻, NO₂⁻, carboxylate, CₑF_{f}SO₃⁻, (where e=2-10 and f=2e+1), acetate, aspartate, benzenesulfonate, benzoate, besylate, bicarbonate, bitartrate, camsylate, carbonate, citrate, decanoate, edetate, esylate, fumarate, gluceptate, gluconate, glutamate, glycolate, glycollyalarsanilate, hexanoate, hydrabamine, hydroxynaphthoate, isthionate, lactate, lactobionate, malate, maleate, mandelate, mesylate, methylbromide, methylnitrate, mucate, napsylate, octanoate, oleate, oxalate, palmitate, pamoate, pantothenate, polygalacturonate, propionate, salicylate, stearate, subacetate, succinate, tartrate, teoclate, tosylate, or triethiiodide. By way of another example, in an embodiment of the disclosure pharmaceutically acceptable salts can comprise a suitable cation selected from aluminum, arginine, benzathine, calcium, chloroprocaine, choline, diethanolamine, ethanolamine, ethylenediamine, lysine, magnesium, histidine, lithium, meglumine, potassium, procaine, sodium, triethylamince, or zinc. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “prodrug” as used herein includes a chemical which may be transformed in vivo to a pharmacologically active drug. The term “metabolite” as used herein includes a chemical that a given agent is transformed into in vivo.

The enantiomeric excess, or “ee,” for a given pair of enantiomers is the percentage of the major enantiomer less the percentage of the minor enantiomer. A “racemate” or “racemic mixture” is an equal mixture of two enantiomers and therefore has 0% ee.

The term “enantiomerically enriched” or “enantioenriched” as used herein includes compounds that are mixtures with one enantiomer's being present in excess over the other (ee >0% and <100%). For example, a sample of 40% ee consists of 70% of the major enantiomer and 30% of the minor enantiomer.

The term “enantiomerically pure” or “enantiopure” as used herein includes compounds where the quantification of the minor enantiomer becomes difficult, e.g., with an ee of 99% or greater. Ideally, enantiopure compounds consist of a single enantiomer only.

The term “sample” as used herein includes any biological specimen obtained from a subject or patient. Samples that can be used in the methods of the present disclosure include, without limitation, tumor sample, whole blood, plasma, serum, red blood cells, white blood cells (e.g., peripheral blood mononuclear cells (PBMC), polymorphonuclear (PMN) cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by random periareolar fine needle aspiration), any other bodily fluid, a tissue sample such as a biopsy (e.g., needle biopsy), and cellular extracts thereof. In some embodiments, when the subject is a pregnant female, the sample may be a fetal DNA sample (e.g., cell-free fetal DNA (cffDNA)).

As used herein, the term “subject” or “patient” refers to mammals and includes, without limitation, human and veterinary animals. In a preferred embodiment, the subject is human. In another preferred embodiment, the subject is a human or an infant.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

An “effective amount” of a compound described herein refers to an amount sufficient to elicit the desired biological response, i.e., treating the state, disorder or condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound described herein is an amount sufficient to provide a therapeutic benefit in the treatment of a state, disorder or condition or to delay or minimize one or more symptoms associated with the state, disorder or condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

In more detail, in one aspect, the present invention refers to a method of treating cancer, autoimmune disease, or any disease involving cellular proliferation in a subject in need thereof, comprising the following steps:
a. determining expression level of 4-hydroxyphenylpyruvate dioxygenase-like (HPDL) in a sample obtained from the subject;
b. comparing the HPDL expression level determined in step (a) with a control level of HPDL expression; and
c. administering an effective amount of an HPDL inhibitor to the subject exhibiting a higher level of HPDL expression as compared to the control level, wherein the HPDL inhibitor is a compound that is ibuprofen or an ibuprofen derivative having the structure of Formula (I), dopamine or a dopamine derivative having the structure of Formula (II), flurbiprofen, ellagic acid, methyldopa, disulfiram, idelalisib, eltrombopag olamine, crystal violet, verteporfin, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations thereof,

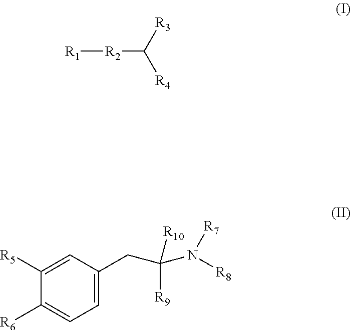

wherein
R₁ is selected from C₁₋₁₂ alkyl, —NH₂, and phenyl optionally substituted by one or more of halogen, —OH, and combinations thereof,
R₂ is selected from C₁₋₆ alkyl and phenyl optionally substituted by one or more of halogen, —OH, and combinations thereof, and
R₃ and R₄ are independently selected from hydrogen, C₁₋₆ alkyl, —NH₂, —CO₂H, phenyl optionally substituted by one or more of halogen, —OH, and combinations thereof,
R₅ and R₆ are independently selected from —OH and —CO₂H;
R₇ and R₈ are independently selected from hydrogen and —NH₂, and
R₉ is selected from hydrogen and —CO₂H,
R₁₀ is selected from hydrogen and C₁₋₆ alkyl,
or a pharmaceutically acceptable salt thereof.

In a specific aspect of the present invention, the compound is ibuprofen or an ibuprofen derivative having the structure of Formula (I) or a pharmaceutically acceptable salt thereof.

In some embodiments, the compound has the structure of Formula (IA):

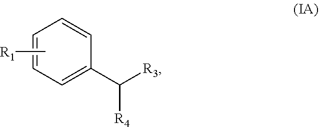

or a pharmaceutically acceptable salt thereof.

In some embodiments, the compound has the structure of Formula (IB):

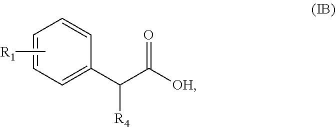

or a pharmaceutically acceptable salt thereof.

In various embodiments of the methods described above, the compound having the structure of Formula (I) is selected from the group consisting of:

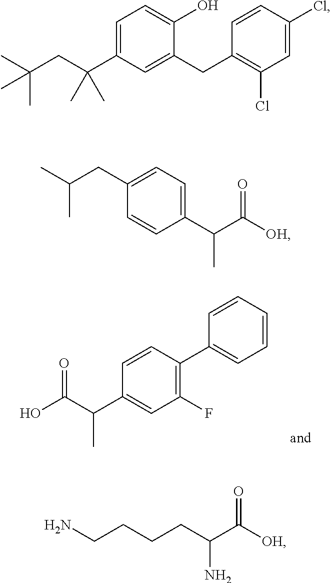

or a pharmaceutically acceptable salt thereof.

In some embodiments, the compound having the structure of Formula (I) is:

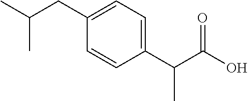

or a pharmaceutically acceptable salt thereof.

In some embodiments, the compound having the structure of Formula (I) is:

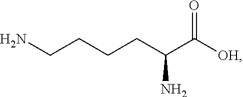

or a pharmaceutically acceptable salt thereof.

In another specific aspect of the present invention, the compound is dopamine or a dopamine derivative having the structure of Formula (II), or a pharmaceutically acceptable salt thereof.

In some embodiments, the compound having the structure of Formula (II) is selected from the group consisting of:

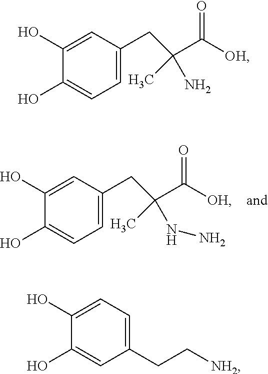

or a pharmaceutically acceptable salt thereof.

In some embodiments, the compound having the structure of Formula (II) is selected from the group consisting of

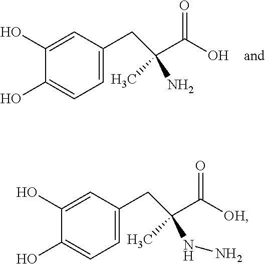

or a pharmaceutically acceptable salt thereof.

In another specific aspect of the present invention, the compound is flurbiprofen, ellagic acid, methyldopa, disulflram, idelalisib, eltrombopag olamine, crystal violet, verteporfn, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations, or a pharmaceutically acceptable salt thereof.

In various embodiments of the methods described above, the sample comprises circulating tumor cells.

In some embodiments, the control level of HPDL expression is determined in normal tissue from the same subject, a sample from a normal subject, or is a predetermined value.

In some embodiments, the expression level of HPDL is determined by determining the level of 4-HMA in the sample.

In various embodiments of the methods described above, the method further comprising administering an additional compound having the structure of Formula (I′):
or a pharmaceutically acceptable salt thereof, wherein:

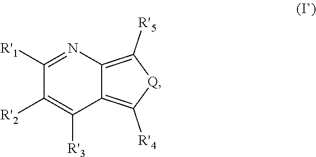

Q is selected from —O—, —NH—, and

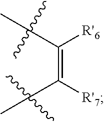

R′₁, R′₂, and R′₃ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂—NHR*, or adjacent two moieties combine to form a fused ring which may optionally contain 1-3 heteroatoms selected from halogen, 0, N, and S and which may be further substituted by one or more R*;
R′₄ and R′₅ are independently selected from —OH and —CO₂H;
R′₆ and R′₇ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂—NHR*, or R₆ and R₇ combine to form a fused ring, and
R* is independently selected at each occurrence from hydrogen or C₁-C₁₂ hydrocarbons each of which optionally contains 1-8 heteroatoms selected from halogen, O, N, and S and combinations thereof.
In some embodiments, Q is

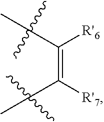

at least one of R′₁, R′₂, and R′₃ is not H.

In some embodiments, the additional compound has the structure of Formula (I′A):

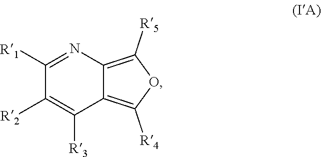

or a pharmaceutically acceptable salt thereof.

In some embodiments, the additional compound has the structure of Formula (I′B):

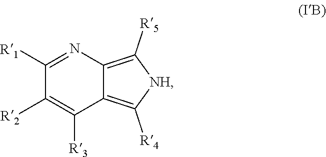

or a pharmaceutically acceptable salt thereof.

In some embodiments, the additional compound has the structure of Formula (I′C):

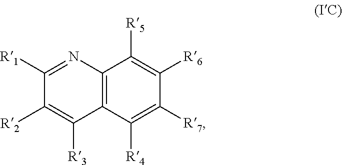

or a pharmaceutically acceptable salt thereof.

In some embodiments, the additional compound has the structure of Formula (I′D):

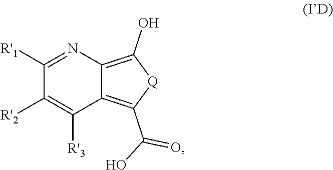

or a pharmaceutically acceptable salt thereof, wherein Q is —O— or —NH—.

In some embodiments, the additional compound has the structure of Formula (I′E):

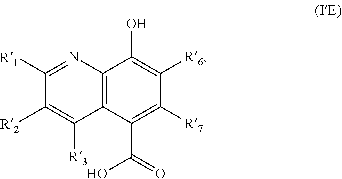

or a pharmaceutically acceptable salt thereof.

In some embodiments, the additional compound having the structure of Formula (I′) is selected from the group consisting of:

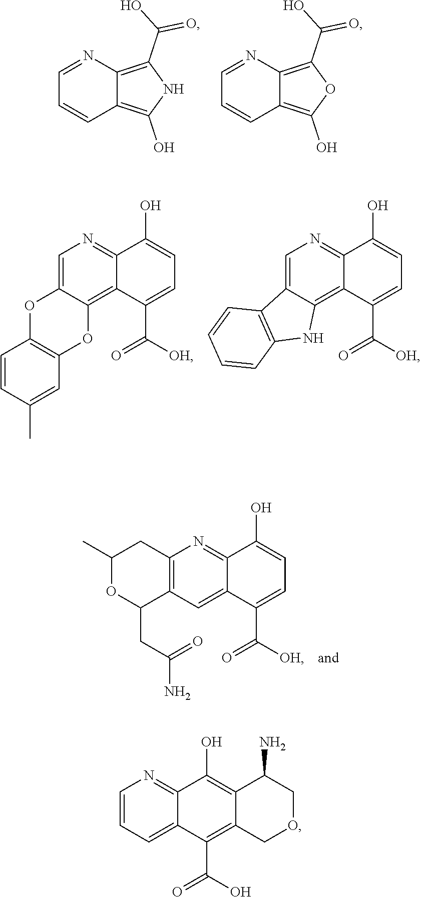

or a pharmaceutically acceptable salt thereof.

In various embodiments of the methods described above, the method further comprising administering an additional compound having the structure of Formula (II′):

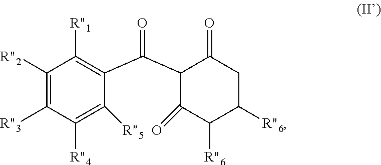

or a pharmaceutically acceptable salt thereof, wherein:
R″₁ is selected from —NO₂, —Cl, and a C₁₋₁₂ alkyl which may be optionally substituted with one or more of —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, and —SO₂—NHR*;
R″₂, R″₃, R″₄, R″₅, R″₆ and R″₇ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂R*, —SO₂—NHR*, or adjacent two moieties combine to form a fused ring which may optionally contain 1-3 heteroatoms selected from halogen, O, N, and S and which may be further substituted by one or more R*, and
R* is independently selected at each occurrence from hydrogen or C₁-C₁₂ hydrocarbons each of which optionally contains 1-8 heteroatoms selected from halogen, O, N, and S and combinations thereof.

In some embodiments, the additional compound does not have the structure selected from

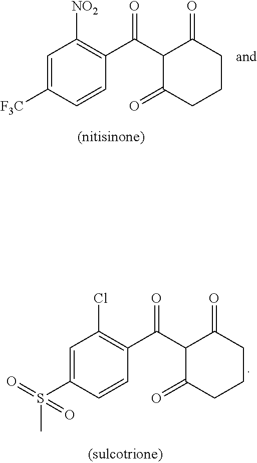 [SMILES: O=C1CCCC(=O)C1C(=O)c1ccc(C(F)(F)F)cc1[N+](=O)[O-] ; CS(=O)(=O)c1ccc(C(=O)C2C(=O)CCCC2=O)c(Cl)c1]

In some embodiments, the additional compound has the structure of Formula (II′A):

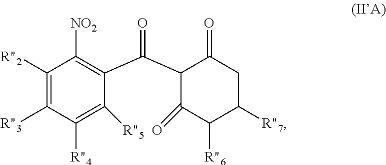

or a pharmaceutically acceptable salt thereof.

In some embodiments, the additional compound has the structure of Formula (II′B):

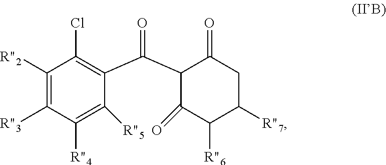

or a pharmaceutically acceptable salt thereof.

In some embodiments, R″₁ is a C₁₋₁₂ alkyl substituted with one or more of —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, and —SO₂—NHR*.

In some embodiments, the additional compound having the structure of Formula (II′) is selected from the group consisting of:

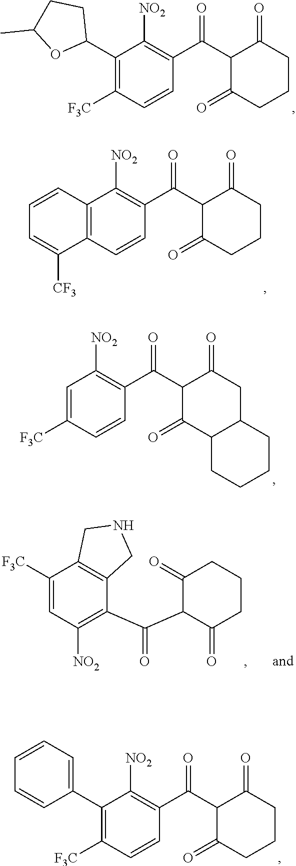 [SMILES: CC1CCC(c2c(C(F)(F)F)ccc(C(=O)C3C(=O)CCCC3=O)c2[N+](=O)[O-])O1 ; O=C1CCCC(=O)C1C(=O)c1ccc2c(C(F)(F)F)cccc2c1[N+](=O)[O-] ; O=C1CC2CCCCC2C(=O)C1C(=O)c1ccc(C(F)(F)F)cc1[N+](=O)[O-] ; O=C1CCCC(=O)C1C(=O)c1c([N+](=O)[O-])cc(C(F)(F)F)c2c1CNC2 ; O=C1CCCC(=O)C1C(=O)c1ccc(C(F)(F)F)c(-c2ccccc2)c1[N+](=O)[O-]]

or a pharmaceutically acceptable salt thereof.

In some embodiments, the additional compound having the structure of Formula (II′) is selected from the group consisting of

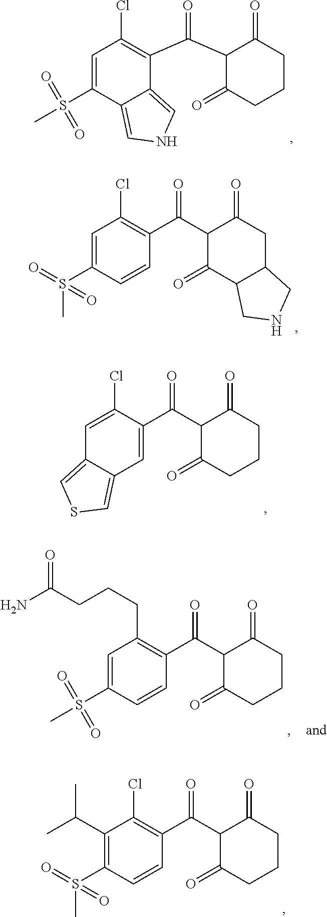

or a pharmaceutically acceptable salt thereof.

In various embodiments of the methods described above, the method further comprising administering N-[5-[[4-[5-[acetyl(hydroxy)amino]pentylamino]-4-oxobutanoyl]-hydroxyamino]pentyl]-N′-(5-aminopentyl)-N′-hydroxybutanediamide (Deferoxamine; DFO).

In various embodiments of the methods described above, the method comprising not administering an HPDL inhibitor to the subject exhibiting a lower or equivalent level of HPDL expression as compared to the control level.

In various embodiments of the methods described above, the method further comprising administering one or more additional treatments to the subject, wherein said additional treatments are selected from a chemotherapy, a chemoradiotherapy, a neoadjuvant chemoradiotherapy, a radiotherapy, a surgery, and any combination thereof.

In some embodiments, the additional treatment is a platinum-based chemotherapy and the method further comprises administering an electron transport chain (ETC) inhibitor.

In some embodiments, the electron transport chain (ETC) inhibitor is metformin, phenformin, BAY84-2243, carboxyamidotriazole, ME344, Fenofibrate, mIBG (meta-iodobenzylguanidine), Alpha-TOS, Lonidamine, Atovaquone, Arsenic trioxide, Nitric Oxide, or Hydrocortisone, or a combination thereof.

In another aspect, provided herein is a method of determining effectiveness of a treatment for cancer, autoimmune disease, or any disease involving cellular proliferation in a subject in a subject, comprising the following steps:
a. determining expression level of 4-hydroxyphenylpyruvate dioxygenase-like (HPDL) in a first sample obtained from the subject before the subject receives a treatment for cancer comprising administering a compound that is ibuprofen or an ibuprofen derivative having the structure of Formula (I), dopamine or a dopamine derivative having the structure of Formula (II), flurbiprofen, ellagic acid, methyldopa, disulfiram, idelalisib, eltrombopag olamine, crystal violet, verteporfin, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations thereof,

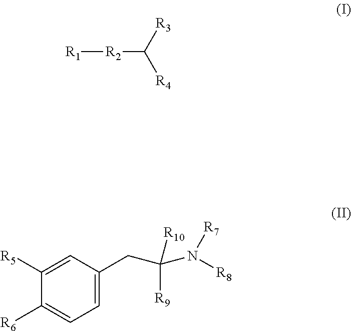

wherein
R₁ is selected from C₁₋₁₂ alkyl, —NH₂, and phenyl optionally substituted by one or more of halogen, —OH, and combinations thereof,
R₂ is selected from C₁₋₆ alkyl and phenyl optionally substituted by one or more of halogen, —OH, and combinations thereof, and
R₃ and R₄ are independently selected from hydrogen, C₁₋₆ alkyl, —NH₂, —CO₂H, phenyl optionally substituted by one or more of halogen, —OH, and combinations thereof,
R₅ and R₆ are independently selected from —OH and —CO₂H;
R₇ and R₈ are independently selected from hydrogen and —NH₂, and
R₉ is selected from hydrogen and —CO₂H,
R₁₀ is selected from hydrogen and methyl,
or a pharmaceutically acceptable salt thereof;
b. determining expression level of HPDL in a second sample obtained from the subject after the subject has received the treatment for cancer, autoimmune disease, or any disease involving cellular proliferation in a subject;
c. comparing the HPDL expression levels determined in the first sample and the second sample; and
d. determining that (i) the treatment is effective if the HPDL expression level in the second sample is lower than HPDL expression level in the first sample, or (ii) the treatment is not effective if the HPDL expression level in the second sample is not lower than the HPDL expression level in the first sample.

In a specific aspect, the treatment for cancer is selected from one or more HPDL inhibitors, a chemotherapy, a chemoradiotherapy, a neoadjuvant chemoradiotherapy, a radiotherapy, a surgery, and any combination thereof.

In another aspect, provided herein is method of predicting prognosis of cancer in a subject having cancer, autoimmune disease, or any disease involving cellular proliferation in a subject, comprising the following steps:
a. determining expression level of 4-hydroxyphenylpyruvate dioxygenase-like (HPDL) protein in a sample obtained from the subject;
b. comparing the HPDL expression level determined in step (a) with a control level of HPDL expression; and
c. determining the subject (i) as having poor prognosis if the HPDL expression level is higher than the control level, or (ii) as having good prognosis if the HPDL expression level is lower than or equal to the control level, wherein if the subject is determined to have poor prognosis, the method further comprising administering to the subject a single-agent chemotherapy, supportive care, and/or a compound that is ibuprofen or an ibuprofen derivative having the structure of Formula (I), dopamine or a dopamine derivative having the structure of Formula (II), flurbiprofen, ellagic acid, methyldopa, disulfiram, idelalisib, eltrombopag olamine, crystal violet, verteporfin, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations thereof,

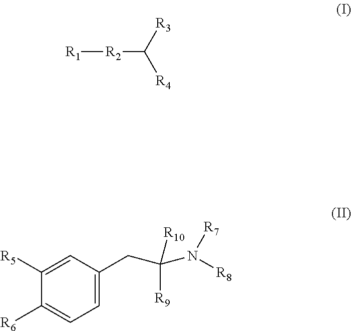

wherein
R₁ is selected from C₁₋₁₂ alkyl, —NH₂, and phenyl optionally substituted by one or more of halogen, —OH, and combinations thereof,
R₂ is selected from C₁₋₆ alkyl and phenyl optionally substituted by one or more of halogen, —OH, and combinations thereof, and
R₃ and R₄ are independently selected from hydrogen, C₁₋₆ alkyl, —NH₂, —CO₂H, phenyl optionally substituted by one or more of halogen, —OH, and combinations thereof,
R₅ and R₆ are independently selected from —OH and —CO₂H;
R₇ and R₈ are independently selected from hydrogen and —NH₂, and
R₉ is selected from hydrogen and —CO₂H,
R₁₀ is selected from hydrogen and methyl,
or a pharmaceutically acceptable salt thereof.

In some embodiments, the control level of HPDL expression is determined in normal tissue from the same subject, a sample from a normal subject, or is a predetermined value.

In various embodiments of the methods described above, the subject has unresectable or borderline respectable pancreatic cancer.

In various embodiments of the methods of predicting prognosis of cancer, autoimmune disease, or any disease involving cellular proliferation as described above, further comprising administering an additional compound having the structure of Formula (I′):

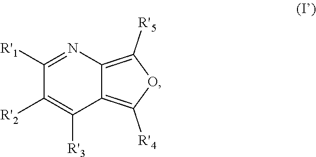

or a pharmaceutically acceptable salt thereof, wherein:
Q is selected from —O—, —NH—, and

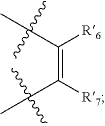

R′₁, R′₂, and R′₃ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂—NHR*, or adjacent two moieties combine to form a fused ring which may optionally contain 1-3 heteroatoms selected from halogen, 0,
N, and S and which may be further substituted by one or more R*;
R′₄ and R′₅ are independently selected from —OH and —CO₂H;
R′₆ and R′₇ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂—NHR*, or R₆ and R₇ combine to form a fused ring, and
R* is independently selected at each occurrence from hydrogen or C₁-C₁₂ hydrocarbons each of which optionally contains 1-8 heteroatoms selected from halogen, O, N, and S and combinations thereof.

In some embodiments of the methods of predicting prognosis of cancer, autoimmune disease, or any disease involving cellular proliferation in a subject having cancer as described above, Q is

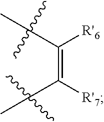

at least one of R′₁, R′₂, and R′₃ is not H.

In some embodiments of the methods of predicting prognosis of cancer, autoimmune disease, or any disease involving cellular proliferation in a subject having cancer as described above, the additional compound has the structure of Formula (I′A):

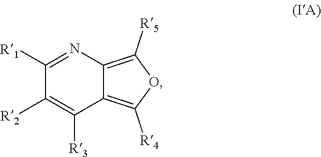

or a pharmaceutically acceptable salt thereof.

In some embodiments, the additional compound has the structure of Formula (I′B):

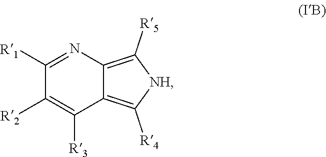

or a pharmaceutically acceptable salt thereof.

In some embodiments, the additional compound has the structure of Formula (I′C):

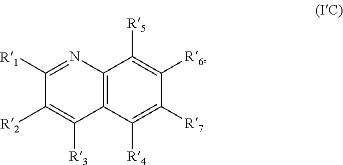

or a pharmaceutically acceptable salt thereof.

In some embodiments, the additional compound has the structure of Formula (I′D):

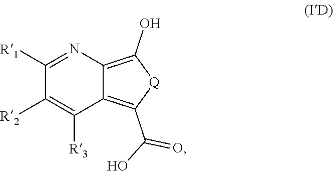

or a pharmaceutically acceptable salt thereof, wherein Q is —O— or —NH—.

In some embodiments, the additional compound has the structure of Formula (I′E):

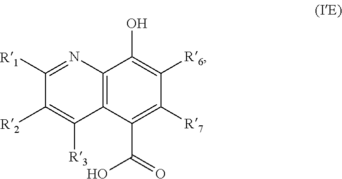

or a pharmaceutically acceptable salt thereof.

In some embodiments, the additional compound having the structure of Formula (I′) is selected from the group consisting of:

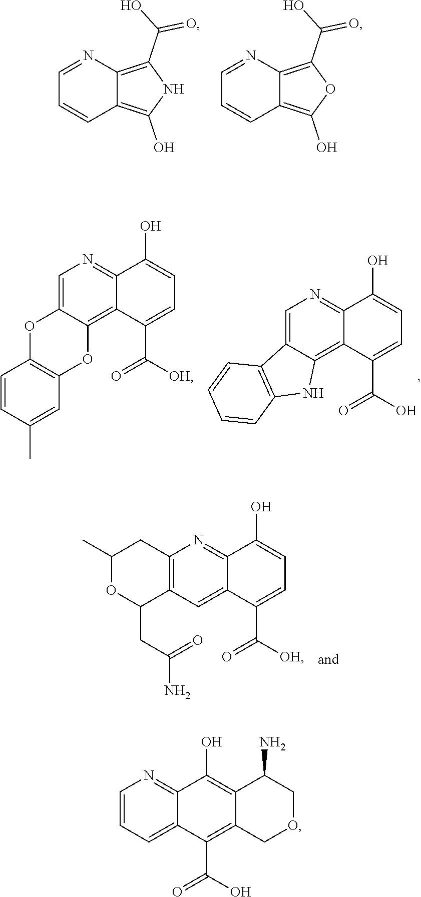

or a pharmaceutically acceptable salt thereof.

In various embodiments of the methods of predicting prognosis of cancer, autoimmune disease, or any disease involving cellular proliferation in a subject having cancer as described above, the method further comprising administering an additional compound having the structure of Formula (II′):

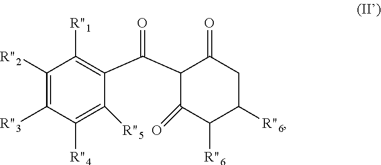

or a pharmaceutically acceptable salt thereof, wherein:
R″₁ is selected from —NO₂, —Cl, and a C₁₋₁₂ alkyl which may be optionally substituted with one or more of —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, and —SO₂—NHR*;
R′₂, R″₃, R″₄, R″₅, R″₆ and R″₇ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂R*, —SO₂—NHR*, or adjacent two moieties combine to form a fused ring which may optionally contain 1-3 heteroatoms selected from halogen, O, N, and S and which may be further substituted by one or more R*, and
R* is independently selected at each occurrence from hydrogen or C₁-C₁₂ hydrocarbons each of which optionally contains 1-8 heteroatoms selected from halogen, O, N, and S and combinations thereof.

In some embodiments, the additional compound does not have the structure selected from

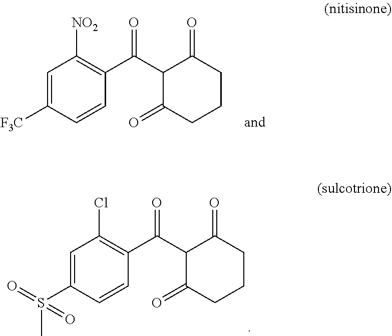 [SMILES: O=C1CCCC(=O)C1C(=O)c1ccc(C(F)(F)F)cc1[N+](=O)[O-] ; CS(=O)(=O)c1ccc(C(=O)C2C(=O)CCCC2=O)c(Cl)c1]

In some embodiments, the additional compound has the structure of Formula (II′A);

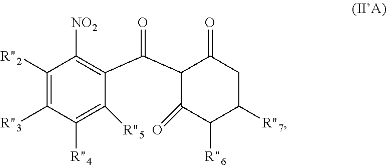

or a pharmaceutically acceptable salt thereof.

In some embodiments, the additional compound has the structure of Formula (II′B):

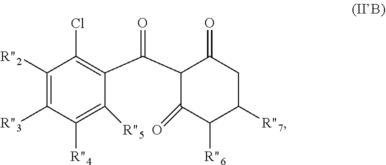

or a pharmaceutically acceptable salt thereof.

In some embodiments, R″₁ is a C₁₋₁₂ alkyl substituted with one or more of —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, and —SO₂—NHR*.

In some embodiments, the additional compound having the structure of Formula (II′) is selected from the group consisting of:

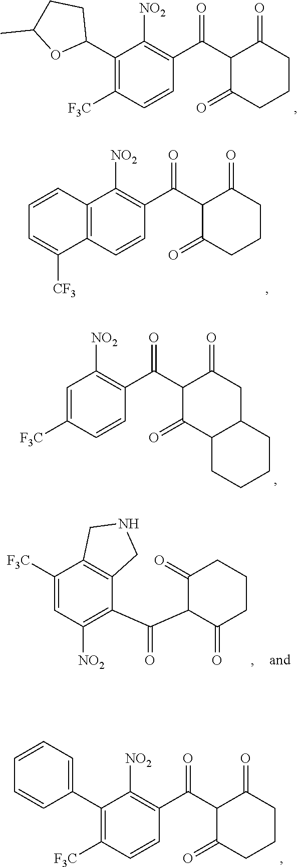 [SMILES: CC1CCC(c2c(C(F)(F)F)ccc(C(=O)C3C(=O)CCCC3=O)c2[N+](=O)[O-])O1 ; O=C1CCCC(=O)C1C(=O)c1ccc2c(C(F)(F)F)cccc2c1[N+](=O)[O-] ; O=C1CC2CCCCC2C(=O)C1C(=O)c1ccc(C(F)(F)F)cc1[N+](=O)[O-] ; O=C1CCCC(=O)C1C(=O)c1c([N+](=O)[O-])cc(C(F)(F)F)c2c1CNC2 ; O=C1CCCC(=O)C1C(=O)c1ccc(C(F)(F)F)c(-c2ccccc2)c1[N+](=O)[O-]]

or a pharmaceutically acceptable salt thereof.

In some embodiments, the additional compound having the structure of Formula (II′) is selected from the group consisting of

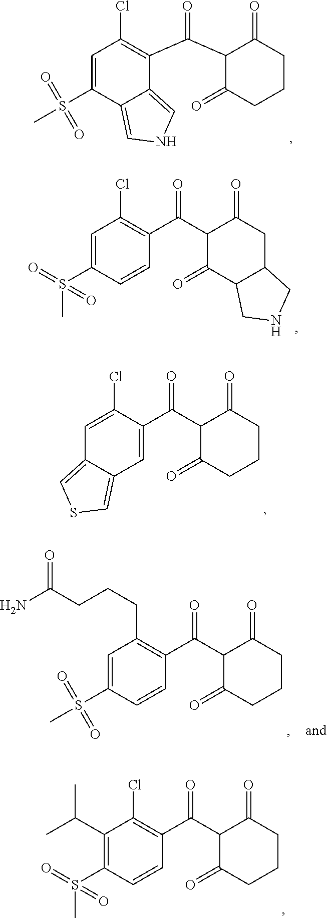

or a pharmaceutically acceptable salt thereof.

In some embodiments of the methods of predicting prognosis of cancer, autoimmune disease, or any disease involving cellular proliferation in a subject having cancer as described above, the method further comprising administering N-[5-[[4-[5-[acetyl(hydroxy)amino]pentylamino]-4-oxobutanoyl]-hydroxyamino]pentyl]-N′-(5-aminopentyl)-N′-hydroxybutanediamide (Deferoxamine; DFO).

In some embodiments of the methods of predicting prognosis of cancer, autoimmune disease, or any disease involving cellular proliferation in a subject having cancer as described above, the method comprising the single-agent chemotherapy is platinum-based chemotherapy. In some embodiments, the method further comprises administering an electron transport chain (ETC) inhibitor in combination with the single-agent chemotherapy. In some embodiments, the electron transport chain (ETC) inhibitor is metformin, phenformin, BAY84-2243, carboxyamidotriazole, ME344, Fenofibrate, mIBG (meta-iodobenzylguanidine), Alpha-TOS, Lonidamine, Atovaquone, Arsenic trioxide, Nitric Oxide, or Hydrocortisone, or a combination thereof.

In some embodiments of the methods of predicting prognosis of cancer, autoimmune disease, or any disease involving cellular proliferation in a subject having cancer as described above, the method further comprising administering to the subject a multiagent chemotherapy, chemoradiotherapy, and/or neoadjuvant chemoradiotherapy if the subject is determined to have good prognosis. In some embodiments, the multiagent chemotherapy is FOLFIRINOX (Leucovorin calcium/Folinic acid, 5-fluorouracil, Irinotecan, Oxaliplatin).

In various embodiments of the methods described above, the HPDL expression level is determined at mRNA level via RNA-seq, reverse transcription polymerase chain reaction (rt-PCR), or fluorescence in situ hybridization (FISH).

In various embodiments of the methods described above, the HPDL expression level is determined at protein level via immunohistochemistry (IHC) staining or immunofluorescence. In some embodiment, the HPDL protein expression level is determined via chromogenic IHC staining using an anti-HPDL antibody as the primary antibody and an HRP-linked secondary antibody followed by DAB staining.

In various embodiments of the methods described above, the cancer for treatment is pancreatic cancer.

In various embodiments of the methods described above, the pancreatic cancer for treatment is pancreatic ductal adenocarcinoma (PDAC) or neuroendocrine pancreatic cancer.

In various embodiments of the methods described above, the compounds disclosed above are used to treat tumors, cancers (e.g., pancreatic and breast cancers), neurodegenerative disorders (e.g., childhood neurodegenerative disorders with spasticity and encephalopathy, and the inherited form of cerebral palsy), autoimmune diseases, and any diseases involving cellular proliferation.

In some embodiments, the compounds disclosed above are used to treat cancers, autoimmune diseases, and any diseases involving cellular proliferation.

In some embodiment, the cancers, autoimmune diseases, and any diseases involving cellular proliferation are HPDL-dependent.

In some embodiment, the compounds disclosed above are used to treat HPDL-dependent tumor.

In some embodiment, the compounds disclosed above are used to treat HPDL-dependent cell proliferation.

In various embodiments of the methods described above, the compounds disclosed above block the synthesis of CoQ10.

In various embodiments of the methods described above, the compounds disclosed above attenuate the growth of HPDL-dependent tumors.

In various embodiments of the methods described above, the compounds disclosed above prevent the proliferation of benign or malignant cells involved in disease processes.

In various embodiments of the methods described above, the subject is a mammal.

In various embodiments of the methods described above, the subject is a human or an infant.

As described above, there is an unmet need for novel therapeutics based on HPDL inhibition for treatment of various diseases and conditions, including HPDL-dependent tumors, cancers (e.g., pancreatic and breast cancers), autoimmune diseases, and any diseases involving unnecessary or uncontrolled cellular proliferation or mitochondrial activation.

Unexpectedly, the present invention found that ibuprofen, dopamine, and related compounds inhibit the dioxygenase HPDL and attenuate the proliferation of HPDL-dependent cells in vitro and in vivo. These results demonstrate that ibuprofen and dopamine, and their derivatives, can block the synthesis of CoQ10. These results also demonstrate that HPDL can be targeted with and inhibited by ibuprofen and dopamine, and their derivatives.

Advantageously, ibuprofen and dopamine, and their derivatives, as HPDL inhibitors, attenuate the growth of HPDL-dependent tumors or cell proliferation. They are of potent and specific HPDL inhibitors that can be of therapeutic value in preventing the proliferation of benign or malignant HPDL-dependent cells involved in disease processes, such cancer, autoimmune disease, or any disease involving cellular proliferation. The diseases also include HPDL-dependent tumors, cancers (e.g., pancreatic and breast cancers), and neurodegenerative disorders (e.g., childhood neurodegenerative disorders with spasticity and encephalopathy, and the inherited form of cerebral palsy).

### EXAMPLES

The following examples are provided to further describe some of the embodiments disclosed herein. The examples are intended to illustrate, not to limit, the disclosed embodiments.

### Experiment 1. Expression and Purification of a Recombinant Version of HPDL Using Insect Cells Expression System

This experiment demonstrates the expression and purification of a recombinant version of HPDL using insect cells expression system. A culture of expiSF9 cells (5×106 viable cells/mL and ≥90% viability) were infected with high-titer virus (human His-tagged HPDL). The infected cells were cultured at 27° C. and then harvest 3 days after infection. Pellets were resuspended in lysis buffer (20 mM Tris, pH 7.5, 200 mM NaCl, 10% glycerol and 5 mM imidazole) and sonicated, and cell debris were pelleted by centrifugation (20,000×g, 30 min). The supernatant was collected and purified using Talon affinity resin (Takara), followed by cation exchange on Akta purifier using a SP HP column (GE Healthcare). Protein purity was assessed via SDS/PAGE and Coomassie staining and pure protein was store at −80° C. at high concentration (>3 mg/ml) in a buffer containing 20 mM Tris, pH 7.5, 200 mM NaCl and 1 mM TCEP.

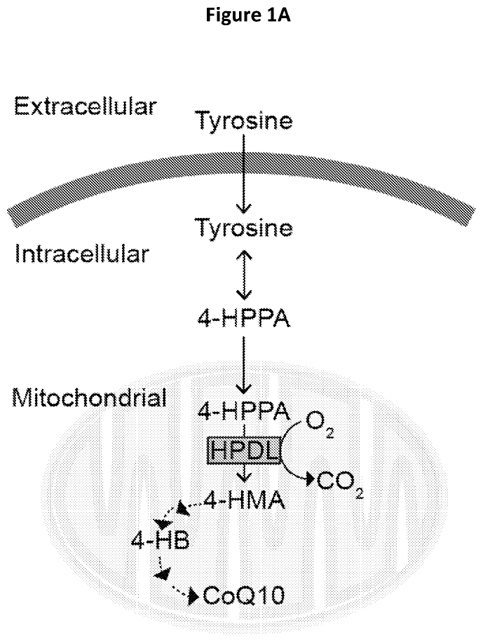

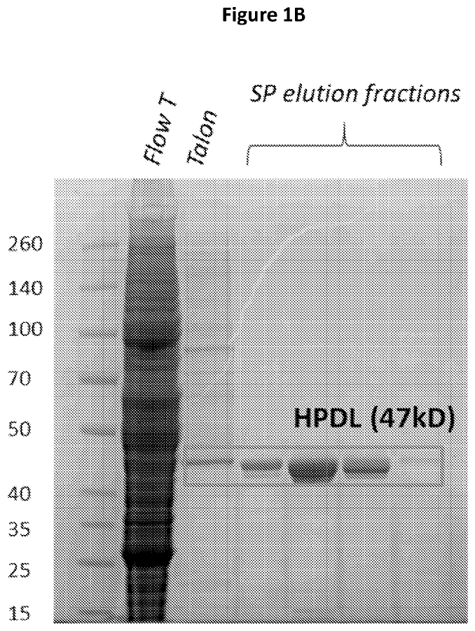

Fractions from HPDL purification run on a Coomassie stained gel (FIG. 1B) show high purity after Talon metal affinity and cation exchange. It is demonstrated that His-tagged HPDL can be produced and purify using expiSF9 expression system.

### Experiment 2. Recombinant HPDL Produced in the Laboratory

This experiment demonstrates that recombinant HPDL produced in the laboratory was catalytically active. His-tagged HPDL (12 ng/L) was incubated with 400 μM of HPPA in assay buffer (20 mM HEPES pH 7.4, 20 μM FeSO4, 0.5 mM sodium ascorbate, and 1 mM Q-mercaptoethanol) for 1 h at 37° C. At the end of the assay, 10 μl of mixture was retrieved and prepared for analysis by gas chromatography-mass spectrometry.

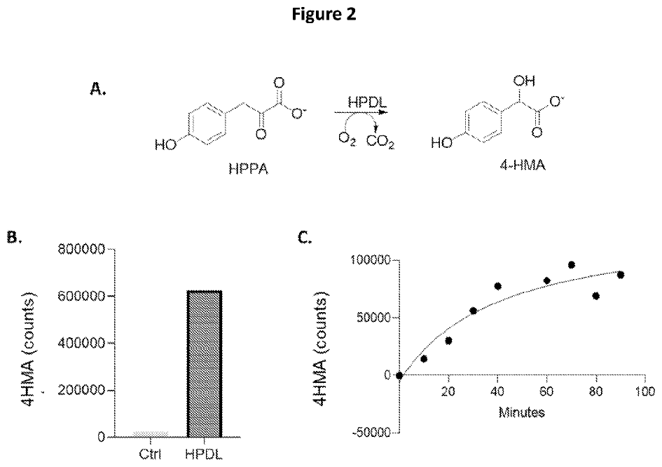

GC-MS detection of 4HMA produced following enzymatic assay on purified enzyme as shown in FIG. 2(B) indicates the high catalytical activity of HPDL. FIG. 2(C) demonstrates GC-MC kinetic characterization of HPDL activity.

### Experiment 3. Evaluation of an FDA Approved Library of Small Molecules by Thermal Shift

This experiment evaluates an FDA approved library of small molecules by thermal shift in order to identify binders of HPDL. Fluorescence based thermal shift assay (FTS) was conducted on Bio-Rad real time PCR detection system. A total of 20 μL/well solution was prepared containing 1 μM HPDL, 2× fluorescent dye (Applied Biosystems, Life Technologies) and small molecules at 10 M concentration in buffer (50 mM HEPES pH 7.5 and 20 μM FeSO4). The plate was heated at 1% of ramp rate (−1.5° C./min) from 25 to 95° C., and Rox was selected as reporter for detection of fluorescence intensity. The melting temperature was recorded for every compound and compared to the reference HPDL melting temperature.

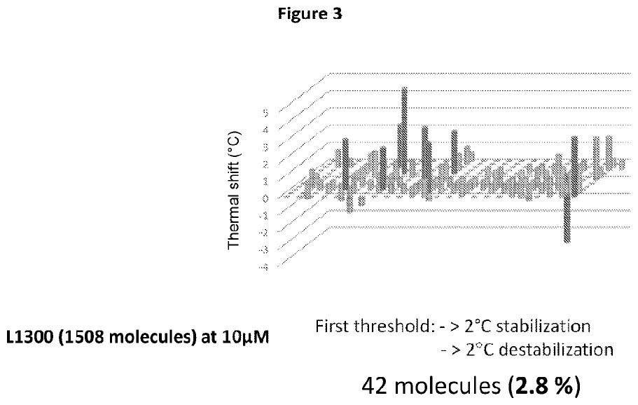

FIG. 3 shows an overview of HPDL thermal stabilization and/or destabilization from a representative panel of compounds after evaluation at 10 μM (blue <2° C. shift, green >2° C. shift). It is demonstrated that SelleckChem FDA approved library contains potential HPDL binders.

### Experiment 4. Evaluation of the Ability of HPDL Binders to Inhibit HPDL Activity

This experiment evaluates the ability of HPDL binders to inhibit its activity. His-tagged HPDL (12 ng/μL) was incubated with 10 μM of HPPA in assay buffer (20 mM HEPES pH 7.4, 20 μM FeSO4, 0.5 mM sodium ascorbate, and 1 mM 0-mercaptoethanol) for 1 h at 37° C. at various inhibitor concentrations. At the end of the assay, 10 μl of mixture was retrieved and prepared for analysis by gas chromatography-mass spectrometry in order to follow 4HMA production.

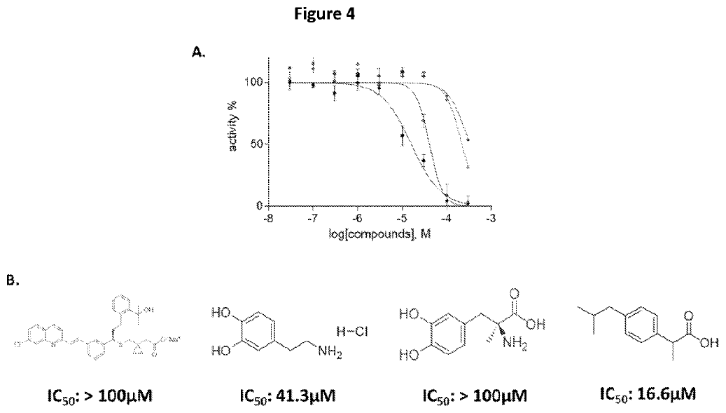

FIG. 4(A) shows dose-response curve of representative binders on HPDL. The four curves in FIG. 4(A) represent, from left to right, ibuprofen, dopamine, montelukast, and methyldopa, respectively. FIG. 4(B) shows chemical structures and IC50 values obtained from these molecules (from left to right in FIG. 4(B), montelukast, dopamine, methyldopa, and ibuprofen). The binders, including ibuprofen, dopamine, and related compounds, are shown to inhibit the dioxygenase HPDL act as HPDL inhibitors.

### Experiment 5. Evaluating the Ability of Ibuprofen to Decreases Cell Growth of HPDL-Dependent Cell Lines in 3D

This experiment evaluates the ability of ibuprofen to decreases cell growth of HPDL-dependent cell lines in 3D. 2,500 cells were seeded into a low-attachment 96-well plate containing DMEM+10% dialysed FBS and 3% Matrigel. Cells were grown for 3 days at 37° C. and 5% CO₂ and ibuprofen (100 uM) was added daily. To measure 3D growth with the CyQUANT Cell Proliferation Assay kit, plates were frozen at −80° C., thawed at room temperature, and equal volumes of 2× CyQUANT reagent+lysis buffer was added to the wells. As a control, wells containing medium and no cells were used as blanks. CyQUANT measurements were performed as described in the manufacturer's instructions.

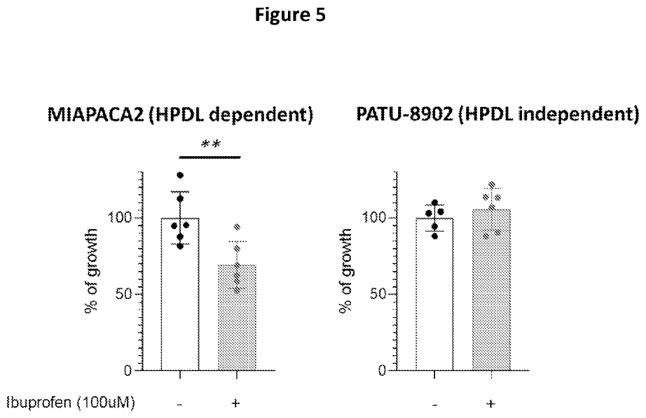

FIG. 5 demonstrates 3D growth assays in MIAPACA2 and PATU-8902 cells after 3 days of treatment with ibuprofen. It is shown that ibuprofen selectively decreases cell growth of HPDL-dependent cell lines in 3D.

### Experiment 6. Evaluating if 4HMA Rescues Ibuprofen-Induced Decreases Cell Growth in 3D

This experiment evaluates if 4HMA rescues ibuprofen-induced decreases cell growth in 3D. 2,500 cells were seeded into a low-attachment 96-well plate containing DMEM+10% dialysed FBS and 3% Matrigel. Cells were grown for 3 days at 37° C. and 5% CO₂ and ibuprofen (100 uM) and/or 4HMA (1 mM) were added daily. To measure 3D growth with the CyQUANT Cell Proliferation Assay kit, plates were frozen at −80° C., thawed at room temperature, and equal volumes of 2×CyQUANT reagent+lysis buffer was added to the wells. Wells containing medium and no cells were used as blanks.

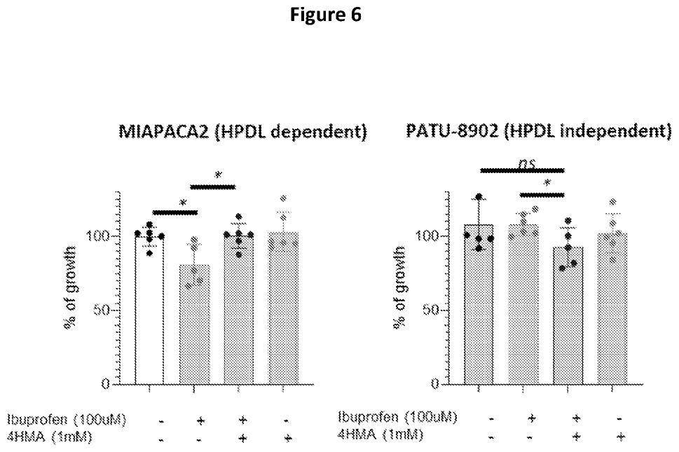

FIG. 6 shows 3D growth assays conducted in MIAPACA2 and PATU-8902 cells after 3 days of treatment with ibuprofen, 4HMA and both molecules. ***P<0.005. It is shown that 4HMA rescues ibuprofen-induced decreases cell growth in 3D.

### Experiment 7. Evaluating if COX Inhibition is Responsible for the Decrease of Cell Growth in 3D

This experiment evaluates if COX inhibition is responsible for the decrease of cell growth in 3D. The ability of aspirin, another COX inhibitor, to bind HPDL was evaluated using thermal shift assay. Fluorescence based thermal shift assay (FTS) was conducted on Bio-Rad real time PCR detection system. A total of 20 μL/well solution was prepared containing 1 μM HPDL, 2× fluorescent dye (Applied Biosystems, Life Technologies) and ibuprofen or aspirin at 10 μM concentration in buffer (50 mM HEPES pH 7.5 and 20 μM FeSO₄). The plate was heated at 1% of ramp rate (˜1.5° C./min) from 25 to 95° C., and Rox was selected as reporter for detection of fluorescence intensity. The melting temperature was recorded for every compound and compared to the reference HPDL melting temperature.

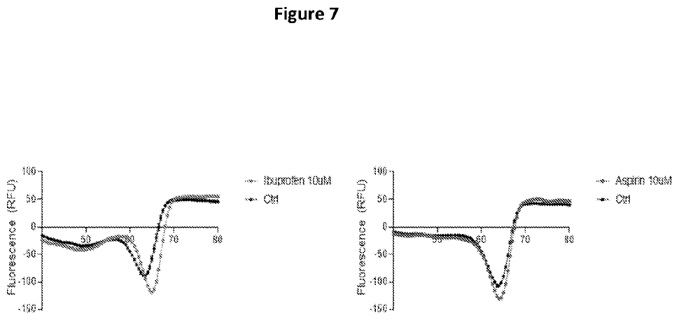

FIG. 7 shows thermal shift curves obtained with a Sypro orange assay of ibuprofen and aspirin on purified HPDL. It is shown that ibuprofen acts as a HPDL binder, while aspirin does not act HPDL binder.

### Experiment 8. Evaluating if COX Inhibition is Responsible for the Decrease of Cell Growth in 3D

This experiment evaluates if COX inhibition is responsible for the decrease of cell growth in 3D. The ability of aspirin, another COX inhibitor, to affect cell growth of HPDL-dependent cell lines in 3D was evaluated. 2,500 cells were seeded into a low-attachment 96-well plate containing DMEM+10% dialysed FBS and 3% Matrigel. Cells were grown for 3 days at 37° C. and 5% CO₂ and ibuprofen (100 uM) or aspirin (100 uM) were added daily. To measure 3D growth with the CyQUANT Cell Proliferation Assay kit, plates were frozen at −80° C., thawed at room temperature, and equal volumes of 2× CyQUANT reagent+lysis buffer was added to the wells. Wells containing medium and no cells were used as blanks.

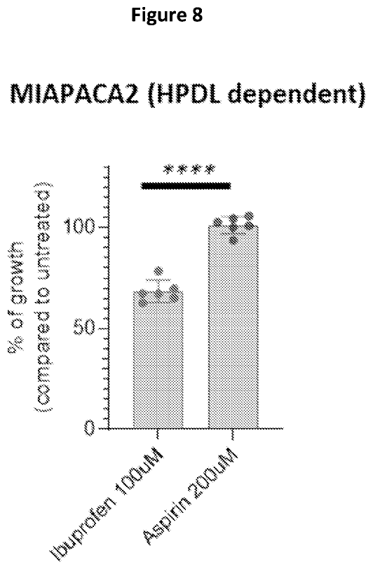

FIG. 8 shows 3D growth assays conducted in MIAPACA2 cells after 3 days of treatment with ibuprofen and aspirin. ***P<0.005. It is shown that aspirin does not affect cell growth of HPDL-dependent cell lines and ibuprofen affects cell growth of HPDL-dependent cell lines in 3D.

### Experiment 9. Evaluating if HPDL Inhibition in Cells is Responsible for the Decrease of Cell Growth in 3D

This experiment evaluates if HPDL inhibition in cells is responsible for the decrease of cell growth in 3D. Ibuprofen was evaluated on MIAPACA2 expressing control or HPDL sgRNA, with or without sgRNA-resistant codon-optimized HPDL (coHPDL) WT and catalytically impaired mutant. 2,500 cells were seeded into a low-attachment 96-well plate containing DMEM+10% dialysed FBS and 3% Matrigel. Cells were grown for 3 days at 37° C. and 5% CO₂ and ibuprofen (100 uM) was added daily. To measure 3D growth with the CyQUANT Cell Proliferation Assay kit, plates were frozen at −80° C., thawed at room temperature, and equal volumes of 2× CyQUANT reagent+lysis buffer was added to the wells. Wells containing medium and no cells were used as blanks.

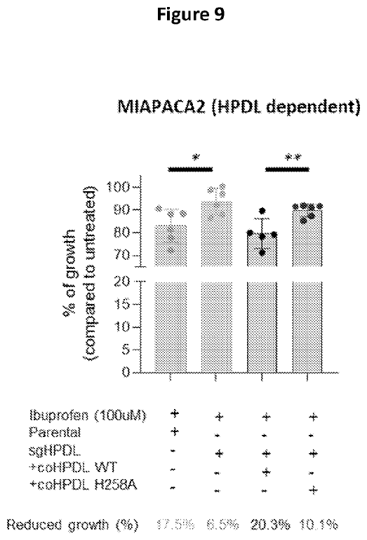

FIG. 9 shows 3D growth assays conducted in MIAPACA2 cells after 3 days of treatment with ibuprofen. ***P<0.005. It is shown that HPDL deletion reduces sensitivity to ibuprofen treatment in 3D.

### Experiment 10. In Vivo Experiments Show that Ibuprofen Supplementation Affects the Growth of HPDL-Dependent PDAC Orthotopic Xenografts and that 4HMA can Rescue this Decrease

This in vivo experiment demonstrate that ibuprofen supplementation affects the growth of HPDL-dependent PDAC orthotopic xenografts and that 4HMA can rescue this decrease. Pancreas orthotopic xenografts were established as follows. In brief, 6-week-old female immunocompromised athymic nude mice (CrTac:NCr-Foxnlnu) were orthotopically injected with tumor cells into the pancreas. Mice were anaesthetized with ketamine (120 mg kg−1) and xylazine (10 mg kg−1) before surgery. MIAPACA2 or PATU-8902 (1×104) were suspended in 20 μl of 50% growth factor-reduced Matrigel (BD Science), and injected into the pancreas. Mice were treated with buprenorphine every 12 h after surgery for 48 h. Mice were treated with ibuprofen (100 mg/kg) and/or 4HMA (10 mg/kg) 3 days after the injection. Both compounds were dissolved into mice water every day to avoid degradation. Mice were sacrificed when tumor development led to significant deterioration of their condition.

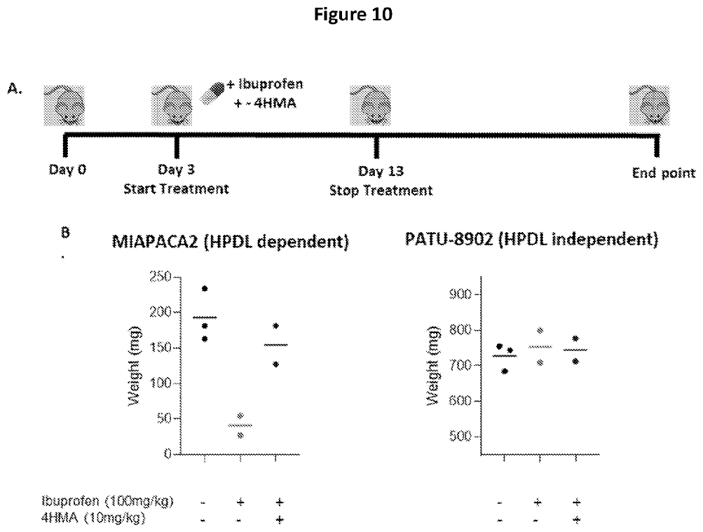

A schematic overview of the in vivo experiment is shown in FIG. 10(A). Ibuprofen and 4HMA were supplemented into mouse drinking water. FIG. 10(B) shows orthotopic pancreatic tumor weight from MIAPACA2 and PATU8902 xenografts after sacrificing the animals. It is shown that ibuprofen affects the growth of HPDL-dependent PDAC orthotopic xenografts.

### Experiment 11. In Vivo Experiments Show that Ibuprofen Supplementation Affects the Development of Pups

This in vivo experiment demonstrates that ibuprofen supplementation affects the development of pups, pups can be rescued by growth of HPDL-dependent PDAC orthotopic xenografts, and that 4HMA addback can rescue this decrease. C₅₇BL/6 pups received daily oral gavage of Ibuprofen (200 mg/kg) and/or 4HMA (10 mg/kg) at different time points during their development (3, 5 and 10 days) and their weight was monitored every day until their death.

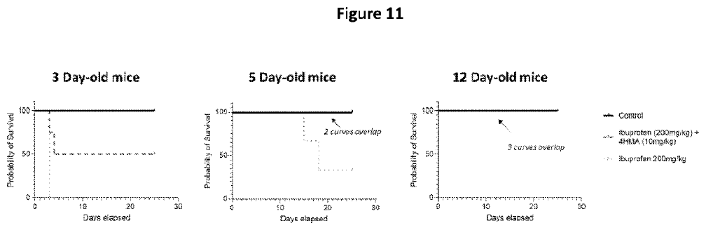

FIG. 11 shows survival plots after daily oral gavage of ibuprofen, 4HMA and both molecules. The treatment was started on 3 different aged pup groups. It is shown that 4HMA supplementation rescues ibuprofen-induced lethality in pups.

### Experiment 12. Investigating the Interaction Pattern of Ibuprofen and HPDL

This experiment investigates the interaction pattern of ibuprofen and HPDL and compares it to HPPA pattern to demonstrate that both compounds show an identical interaction pattern with the enzyme. Ibuprofen and HPPA were docked into the catalytic cavity of human HPDL (Alphafold predictive model) using MOE (Chemical Computing Group) software. Their interaction pattern was analyzed and the key residues interaction with both compounds were highlighted. A global docking score (S score, MOE computing prediction) was obtained for both molecules.

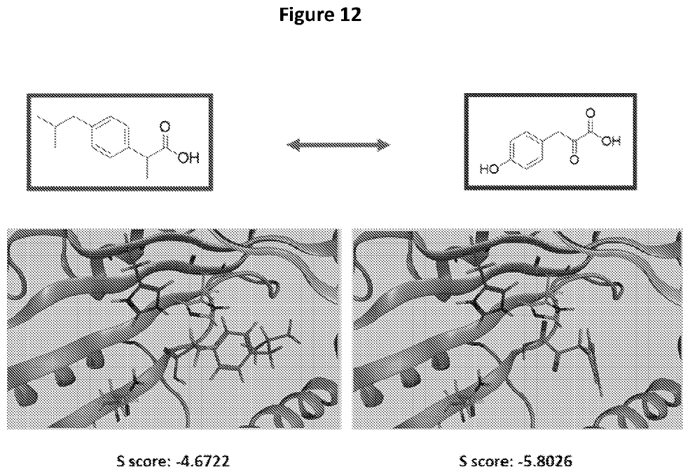

FIG. 12 shows docking simulation of ibuprofen and HPPA in HPDL active site with three residues interacting with the compound (H258, H163 and Q324). Alphafold predicted model is used for HPDL. It is shown that ibuprofen and HPPA show an identical interaction pattern with HPDL.

The above experiments demonstrate that ibuprofen, dopamine, and related compounds inhibit the dioxygenase HPDL and attenuate the proliferation of HPDL-dependent cells in vitro and in vivo.

### REFERENCES

1. Morton R A. Ubiquinone. Nature. Nature Publishing Group; 1958 Dec. 27; 182(4652):1764-7.
2. Quiles J L, Ochoa J J, Ramirez-Tortosa M C, Linde J, Bompadre S, Battino M, et al. Coenzyme Q concentration and total antioxidant capacity of human milk at different stages of lactation in mothers of preterm and full-term infants. Free Radic Res. 2006 February; 40(2):199-206.
3. Gutman M. Electron flux through the mitochondrial ubiquinone. Biochim Biophys Acta. 1980 Dec. 22; 594(1):53-84.
4. Bentinger M, Dallner G, Chojnacki T, Swiezewska E. Distribution and breakdown of labeled coenzyme Q10 in rat. Free Radic Biol Med. 2003 Mar. 1; 34(5):563-75.
5. Bentinger M, Tekle M, Brismar K, Chojnacki T, Swiezewska E, Dallner G. Stimulation of coenzyme Q synthesis. Biofactors. 2008; 32(1-4):99-111.
6. Tekle M, Bentinger M, Nordman T, Appelkvist E-L, Chojnacki T, Olsson J M. Ubiquinone biosynthesis in rat liver peroxisomes. Biochem Biophys Res Commun. 2002 Mar. 15; 291(5):1128-33.
7. Faust J R, Goldstein J L, Brown M S. Synthesis of ubiquinone and cholesterol in human fibroblasts: regulation of a branched pathway. Arch Biochem Biophys [Internet]. 1979 January; 192(1):86-99. Retrieved from: https://linkinghub.elsevier.com/retrieve/pii/0003986179900742
8. Taylor B A. Does Coenzyme Q10 Supplementation Mitigate Statin-Associated Muscle Symptoms?Pharmacological and Methodological Considerations. Am J Cardiovasc Drugs. Springer International Publishing; 2018 April; 18(2):75-82.
9. Stefely J A, Pagliarini D J. Biochemistry of Mitochondrial Coenzyme Q Biosynthesis. Trends Biochem Sci. 2017 October; 42(10):824-43. PMCID: PMC5731490
10. Forsgren M, Attersand A, Lake S, Gronler J, Swiezewska E, Dallner G, et al. Isolation and functional expression of human COQ2, a gene encoding a polyprenyl transferase involved in the synthesis of CoQ. Biochem J. 2004 Sep. 1; 382(Pt 2):519-26. PMCID: PMC1133808
11. Payet L-A, Leroux M, Willison J C, Kihara A, Pelosi L, Pierrel F. Mechanistic Details of Early Steps in Coenzyme Q Biosynthesis Pathway in Yeast. Cell Chem Biol. 2016 Oct. 20; 23(10):1241-50.
12. Acosta M J, Vazquez Fonseca L, Desbats M A, Cerqua C. Zordan R, Trevisson E, et al. Coenzyme Q biosynthesis in health and disease. Biochim Biophys Acta. 2016 August; 1857(8):1079-85.
13. Hirano M, Garone C, Quinzii C M. CoQ(10) deficiencies and MNGIE: two treatable mitochondrial disorders. Biochim Biophys Acta. 2012 May; 1820(5):625-31. PMCID: PMC3319470
14. Birsoy K, Wang T, Chen W W, Freinkman E, Abu-Remaileh M, Sabatini D M. An Essential Role of the Mitochondrial Electron Transport Chain in Cell Proliferation Is to Enable Aspartate Synthesis. Cell [Internet]. 2015 Jul. 30; 162(3):540-51. Retrieved from: http://dx.doi.org/10.1016/j.cell.2015.07.016
15. Sullivan L B, Gui D Y, Hosios A M, Bush L N, Freinkman E, Vander Heiden M G. Supporting Aspartate Biosynthesis Is an Essential Function of Respiration in Proliferating Cells. Cell. Elsevier; 2015 Jul. 30; 162(3):552-63. PMCID: PMC4522278
16. Banh R S, Kim E S, Spillier Q. Biancur D E, Yamamoto K, Sohn A S W, et al. The polar oxy-metabolome reveals the 4-hydroxymandelate CoQ10 synthesis pathway. Nature. 2021 September; 597(7876):420-5.
17. Husain R A, Grimmel M, Wagner M, Hennings J C, Marx C, Feichtinger R G, et al. Bi-allelic HPDL Variants Cause a Neurodegenerative Disease Ranging from Neonatal Encephalopathy to Adolescent-Onset Spastic Paraplegia. Am J Hum Genet. 2020 Aug. 6; 107(2):364-73. PMCID: PMC7413886
18. Ghosh S G, Lee S, Fabunan R, Chai G, Zaki M S, Abdel-Salam G, et al. Biallelic variants in HPDL, encoding 4-hydroxyphenylpyruvate dioxygenase-like protein, lead to an infantile neurodegenerative condition. Genet Med. 2021 March; 23(3):524-33.
19. Morgan N V, Yngvadottir B, O'Driscoll M, Clark G R, Walsh D, Martin E, et al. Evidence that autosomal recessive spastic cerebral palsy-1 (CPSQ1) is caused by a missense variant in HPDL. Brain Commun. 2021; 3(1):fcab002. PMCID: PMC7892364
20. Wiessner M, Maroofian R, Ni M-Y, Pedroni A, Müller J S, Stucka R, et al. Biallelic variants in HPDL cause pure and complicated hereditary spastic paraplegia. Brain [Internet]. 2021 Jun. 22; 144(5):1422-34. Retrieved from: http://eutils.ncbi.nlm.nih.gov/entrez/eutils/elink.fcgi?dbfrom=pubmed&id=339702 00&retmode=ref&cmd=prlinks. PMCID: PMC8219359
21. Ye X, Wei X, Liao J, Chen P, Li X, Chen Y, et al. 4-Hydroxyphenylpyruvate Dioxygenase-Like Protein Promotes Pancreatic Cancer Cell Progression and Is Associated With Glutamine-Mediated Redox Balance. Front Oncol. 2020; 10:617190. PMCID: PMC7848781

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

What is claimed is:

1. A method of treating cancer, autoimmune disease, or any disease involving cellular proliferation in a subject in need thereof, comprising the following steps:
a. determining expression level of 4-hydroxyphenylpyruvate dioxygenase-like (HPDL) in a sample obtained from the subject;
b. comparing the HPDL expression level determined in step (a) with a control level of HPDL expression; and
c. administering an effective amount of an HPDL inhibitor to the subject exhibiting a higher level of HPDL expression as compared to the control level,
wherein the HPDL inhibitor is a compound that is ibuprofen or an ibuprofen derivative having the structure of Formula (I), dopamine or a dopamine derivative having the structure of Formula (TT), flurbiprofen, ellagic acid, methyldopa, disulfiram, idelalisib, eltrombopag olamine, crystal violet, verteporfin, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations thereof, 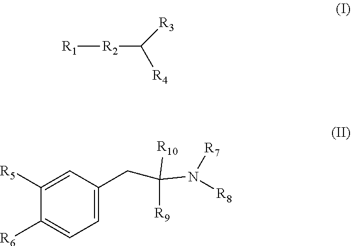
wherein
R₁ is selected from C₁₋₁₂ alkyl, —NH₂, and phenyl optionally substituted by one or more of halogen, —OH, and combinations thereof,
R₂ is selected from C₁₋₆ alkyl and phenyl optionally substituted by one or more of halogen, —OH, and combinations thereof, and
R₃ and R₄ are independently selected from hydrogen, C₁₋₆ alkyl, —NH₂, —CO₂H, phenyl optionally substituted by one or more of halogen, —OH, and combinations thereof,
R₅ and R₆ are independently selected from —OH and —CO₂H;
R₇ and R₈ are independently selected from hydrogen and —NH₂, and
R₉ is selected from hydrogen and —CO₂H,
R₁₀ is selected from hydrogen and C₁₋₆ alkyl,
or a pharmaceutically acceptable salt thereof.

2. The method of claim 1, wherein the compound is ibuprofen or an ibuprofen derivative having the structure of Formula (I) or a pharmaceutically acceptable salt thereof.

3. The method of claim 2, wherein the compound has the structure of Formula (IA): 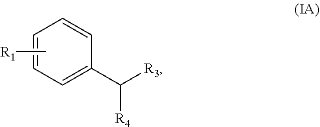
or a pharmaceutically acceptable salt thereof.

4. The method of claim 4, wherein the compound has the structure of Formula (IB): 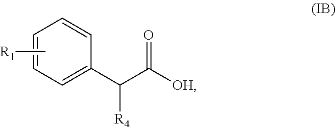
or a pharmaceutically acceptable salt thereof.

5. The method of any one of claims 1 and 1, where the compound having the structure of Formula (I) is selected from the group consisting of 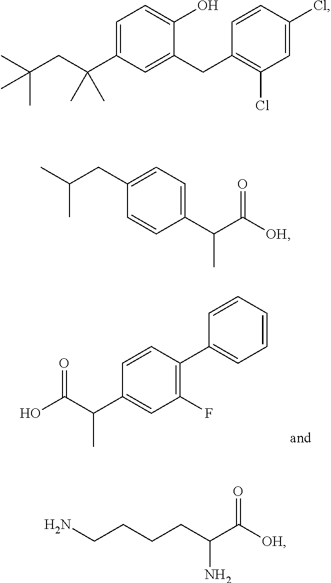
or a pharmaceutically acceptable salt thereof.

6. The method of any one of claims 1-2 and 5, where the compound having the structure of Formula (I) is: 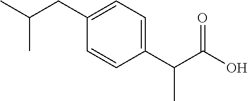
or a pharmaceutically acceptable salt thereof.

7. The method of any one of claims 1-4, where the compound having the structure of Formula (I) is: 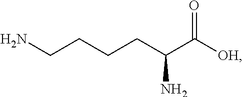
or a pharmaceutically acceptable salt thereof.

8. The method of claim 1, wherein the compound is dopamine or a dopamine derivative having the structure of Formula (II), or a pharmaceutically acceptable salt thereof.

9. The method of any one of claims 1 and 8, where the compound having the structure of Formula (II) is selected from the group consisting of: 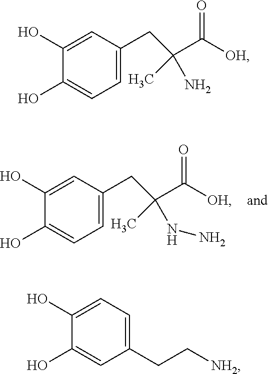
or a pharmaceutically acceptable salt thereof.

10. The method of any one of claims 1 and 8-9, where the compound having the structure of Formula (II) is selected from the group consisting of 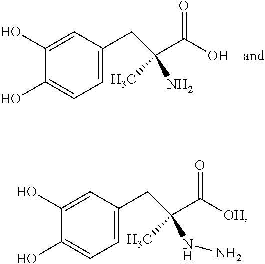
or a pharmaceutically acceptable salt thereof.

11. The method of claim 1, wherein the compound is flurbiprofen, ellagic acid, methyldopa, disulfiram, idelalisib, eltrombopag olamine, crystal violet, verteporfin, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations, or a pharmaceutically acceptable salt thereof.

12. The method of claim 1, wherein the sample comprises circulating tumor cells.

13. The method of any one of claims 1-12, wherein the control level of HPDL expression is determined in normal tissue from the same subject, a sample from a normal subject, or is a predetermined value.

14. The method of any one of claims 1-13, wherein the expression level of HPDL is determined by determining the level of 4-HMA in the sample.

15. The method of any one of claims 1-14, further comprising administering an additional compound having the structure of Formula (I′): 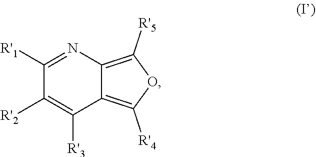
or a pharmaceutically acceptable salt thereof, wherein:
Q is selected from —O—, —NH—, and 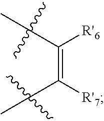
R′₁, R′₂, and R′₃ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂—NHR*, or adjacent two moieties combine to form a fused ring which may optionally contain 1-3 heteroatoms selected from halogen, O, N, and S and which may be further substituted by one or more R*;
R′₄ and R's are independently selected from —OH and —CO₂H;
R′₆ and R′₇ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂—NHR*, or R₆ and R₇ combine to form a fused ring, and
R* is independently selected at each occurrence from hydrogen or C₁-C₁₂ hydrocarbons each of which optionally contains 1-8 heteroatoms selected from halogen, O, N, and S and combinations thereof.

16. The method of claim 15, wherein when Q is 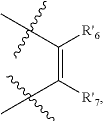
at least one of R′₁, R′₂, and R′₃ is not H.

17. The method of claim 15, wherein the additional compound has the structure of Formula (IA): 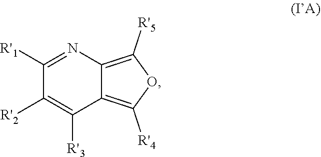
or a pharmaceutically acceptable salt thereof.

18. The method of claim 15, wherein the additional compound has the structure of Formula (I′B): 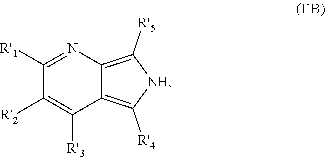
or a pharmaceutically acceptable salt thereof.

19. The method of claim 15, wherein the additional compound has the structure of Formula (I′C): 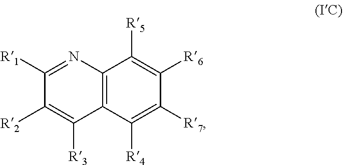
or a pharmaceutically acceptable salt thereof.

20. The method of claim 15, wherein the additional compound has the structure of Formula (I′D): 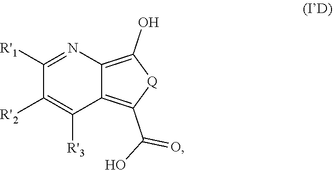
or a pharmaceutically acceptable salt thereof, wherein Q is —O— or —NH—.

21. The method of claim 15, wherein the additional compound has the structure of Formula (I′E): 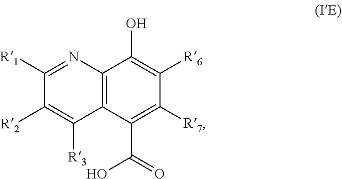
or a pharmaceutically acceptable salt thereof.

22. The method of claim 15, wherein the additional compound having the structure of Formula (I′) is selected from the group consisting of: 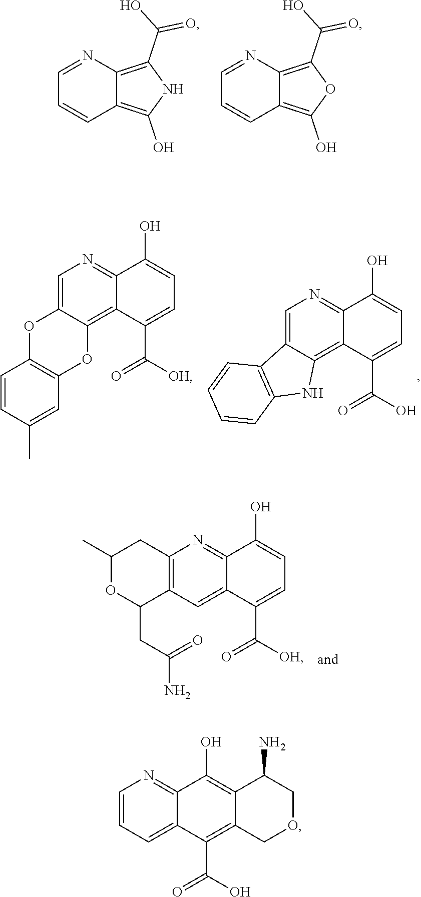
or a pharmaceutically acceptable salt thereof.

23. The method of claim 1-14, further comprising administering an additional compound having the structure of Formula (II′): 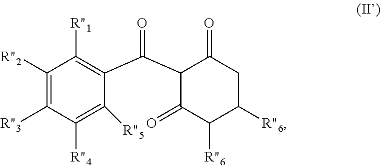
or a pharmaceutically acceptable salt thereof, wherein:
R″₁ is selected from —NO₂, —Cl, and a C₁₋₁₂ alkyl which may be optionally substituted with one or more of —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, and —SO₂—NHR*;
R″₂, R″₃, R″₄, R″₅, R″₆ and R″₇ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂R*, —SO₂—NHR*, or adjacent two moieties combine to form a fused ring which may optionally contain 1-3 heteroatoms selected from halogen, O, N, and S and which may be further substituted by one or more R*, and
R* is independently selected at each occurrence from hydrogen or C₁-C₁₂ hydrocarbons each of which optionally contains 1-8 heteroatoms selected from halogen, O, N, and S and combinations thereof.

24. The method of claim 23, wherein the additional compound does not have the structure selected from

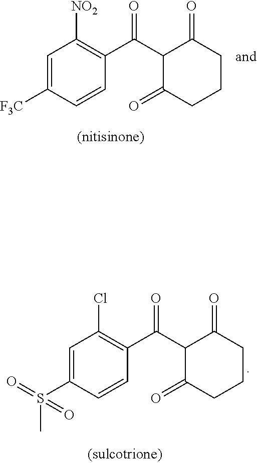 [SMILES: O=C1CCCC(=O)C1C(=O)c1ccc(C(F)(F)F)cc1[N+](=O)[O-] ; CS(=O)(=O)c1ccc(C(=O)C2C(=O)CCCC2=O)c(Cl)c1]

25. The method of claim 23, wherein the additional compound has the structure of Formula (II′A): 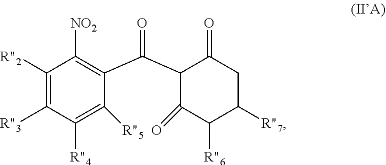
or a pharmaceutically acceptable salt thereof.

26. The method of claim 23, wherein the additional compound has the structure of Formula (II′B): 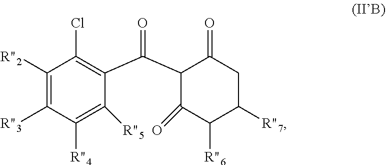
or a pharmaceutically acceptable salt thereof.

27. The method of claim 23, wherein R″₁ is a C₁₋₁₂ alkyl substituted with one or more of —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, and —SO₂—NHR*.

28. The method of claim 23, wherein the additional compound having the structure of Formula (II′) is selected from the group consisting of 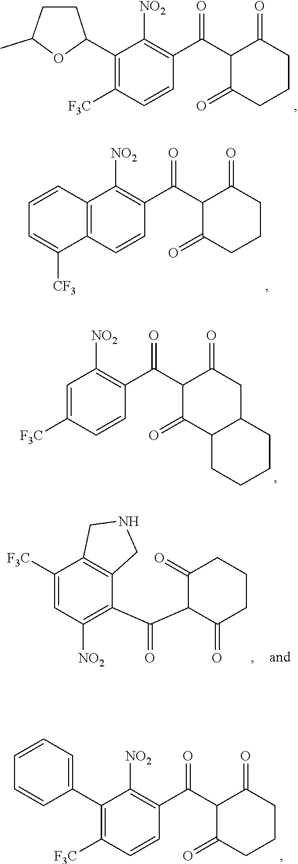 [SMILES: CC1CCC(c2c(C(F)(F)F)ccc(C(=O)C3C(=O)CCCC3=O)c2[N+](=O)[O-])O1 ; O=C1CCCC(=O)C1C(=O)c1ccc2c(C(F)(F)F)cccc2c1[N+](=O)[O-] ; O=C1CC2CCCCC2C(=O)C1C(=O)c1ccc(C(F)(F)F)cc1[N+](=O)[O-] ; O=C1CCCC(=O)C1C(=O)c1c([N+](=O)[O-])cc(C(F)(F)F)c2c1CNC2 ; O=C1CCCC(=O)C1C(=O)c1ccc(C(F)(F)F)c(-c2ccccc2)c1[N+](=O)[O-]]
or a pharmaceutically acceptable salt thereof.

29. The method of claim 23, wherein the additional compound having the structure of Formula (II′) is selected from the group consisting of 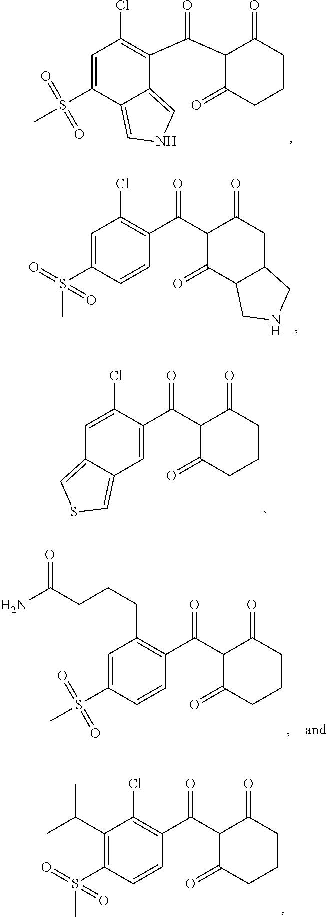
or a pharmaceutically acceptable salt thereof.

30. The method of any one of claims 1-14, further comprising administering N-[5-[[4-[5-[acetyl(hydroxy)amino]pentylamino]-4-oxobutanoyl]-hydroxyamino]pentyl]-N-(5-aminopentyl)-N-hydroxybutanediamide (Deferoxamine; DFO).

31. The method of any one of claims 1-30, wherein the method comprises not administering an HPDL inhibitor to the subject exhibiting a lower or equivalent level of HPDL expression as compared to the control level.

32. The method of any one of claims 1-31, further comprising administering one or more additional treatments to the subject, wherein said additional treatments are selected from a chemotherapy, a chemoradiotherapy, a neoadjuvant chemoradiotherapy, a radiotherapy, a surgery, and any combination thereof.

33. The method of claim 32, wherein the additional treatment is a platinum-based chemotherapy and the method further comprises administering an electron transport chain (ETC) inhibitor.

34. The method of claim 33, wherein the electron transport chain (ETC) inhibitor is metformin, phenformin, BAY84-2243, carboxyamidotriazole, ME344, Fenofibrate, mIBG (meta-iodobenzylguanidine), Alpha-TOS, Lonidamine, Atovaquone, Arsenic trioxide, Nitric Oxide, or Hydrocortisone, or a combination thereof.

35. A method of determining effectiveness of a treatment for cancer, autoimmune disease, or any disease involving cellular proliferation in a subject, comprising the following steps:
a. determining expression level of 4-hydroxyphenylpyruvate dioxygenase-like (HPDL) in a first sample obtained from the subject before the subject receives a treatment for cancer comprising administering a compound that is ibuprofen or an ibuprofen derivative having the structure of Formula (I), dopamine or a dopamine derivative having the structure of Formula (II), flurbiprofen, ellagic acid, methyldopa, disulfiram, idelalisib, eltrombopag olamine, crystal violet, verteporfin, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations thereof, 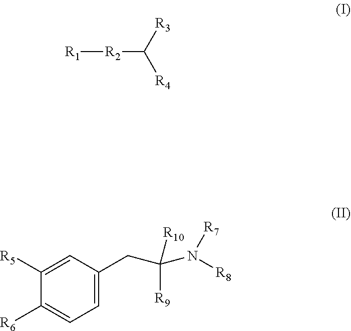
wherein
R₁ is selected from C₁₋₁₂ alkyl, —NH₂, and phenyl optionally substituted by one or more of halogen, —OH, and combinations thereof,
R₂ is selected from C₁₋₆ alkyl and phenyl optionally substituted by one or more of halogen, —OH, and combinations thereof, and
R₃ and R₄ are independently selected from hydrogen, C₁₋₆ alkyl, —NH₂, —CO₂H, phenyl optionally substituted by one or more of halogen, —OH, and combinations thereof,
R₅ and R₆ are independently selected from —OH and —CO₂H;
R₇ and R₈ are independently selected from hydrogen and —NH₂, and
R₉ is selected from hydrogen and —CO₂H,
R₁₀ is selected from hydrogen and methyl,
or a pharmaceutically acceptable salt thereof;
b. determining expression level of HPDL in a second sample obtained from the subject after the subject has received the treatment for cancer, autoimmune disease, or any disease involving cellular proliferation;
c. comparing the HPDL expression levels determined in the first sample and the second sample; and
d. determining that (i) the treatment is effective if the HPDL expression level in the second sample is lower than HPDL expression level in the first sample, or (ii) the treatment is not effective if the HPDL expression level in the second sample is not lower than the HPDL expression level in the first sample.

36. The method claim 35, wherein the treatment for cancer is selected from one or more HPDL inhibitors, a chemotherapy, a chemoradiotherapy, a neoadjuvant chemoradiotherapy, a radiotherapy, a surgery, and any combination thereof.

37. A method of predicting prognosis of cancer in a subject having cancer, autoimmune disease, or any disease involving cellular proliferation, comprising the following steps:
a. determining expression level of 4-hydroxyphenylpyruvate dioxygenase-like (HPDL) protein in a sample obtained from the subject;
b. comparing the HPDL expression level determined in step (a) with a control level of HPDL expression; and
c. determining the subject (i) as having poor prognosis if the HPDL expression level is higher than the control level, or (ii) as having good prognosis if the HPDL expression level is lower than or equal to the control level, wherein if the subject is determined to have poor prognosis, the method further comprising administering to the subject a single-agent chemotherapy, supportive care, and/or a compound that is ibuprofen or an ibuprofen derivative having the structure of Formula (I), dopamine or a dopamine derivative having the structure of Formula (II), flurbiprofen, ellagic acid, methyldopa, disulfiram, idelalisib, eltrombopag olamine, crystal violet, verteporfin, pioglitazone HCl, rosiglitazone HCl, enoxolone, triamcinolone, tideglusib, tolcapone, thimerosal, zinc pyrithione, montelukast sodium, nitroxoline, carbenoxolone sodium, sildenafil mesylate, saxagliptin hydrate, fenoldopam mesylate, gallic acid, clofoctol, hexachlorophene, cetylpyridinium chloride, povidone iodine, bithionol, bronopol, bardoxolone methyl, oltipraz, troglitazone, cangrelor tetrasodium, bismuth subcitrate potassium, puromycin, ledipasvir, obeticholic acid, erlotinib, tenofovir alafenamide, entrectinib, venetoclaxthe, or combinations thereof, 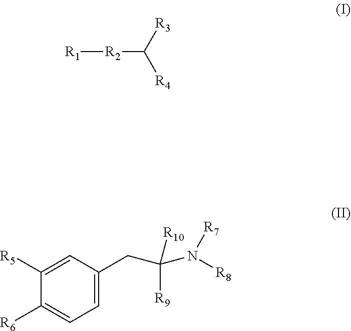
wherein
R₁ is selected from C₁₋₁₂ alkyl, —NH₂, and phenyl optionally substituted by one or more of halogen, —OH, and combinations thereof,
R₂ is selected from C₁₋₆ alkyl and phenyl optionally substituted by one or more of halogen, —OH, and combinations thereof, and
R₃ and R₄ are independently selected from hydrogen, C₁₋₆ alkyl, —NH₂, —CO₂H, phenyl optionally substituted by one or more of halogen, —OH, and combinations thereof,
R₅ and R₆ are independently selected from —OH and —CO₂H;
R₇ and R₈ are independently selected from hydrogen and —NH₂, and
R₉ is selected from hydrogen and —CO₂H,
R₁₀ is selected from hydrogen and methyl,
or a pharmaceutically acceptable salt thereof.

38. The method of claim 37, wherein the control level of HPDL expression is determined in normal tissue from the same subject, a sample from a normal subject, or is a predetermined value.

39. The method of claim 37 or 38, wherein the subject has unresectable or borderline respectable pancreatic cancer.

40. The method of claim 37, wherein the method further comprising administering an additional compound having the structure of Formula (T′): 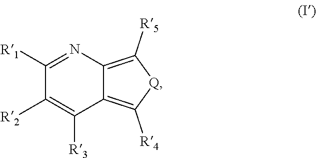
or a pharmaceutically acceptable salt thereof, wherein:
Q is selected from —O—, —NH—, and 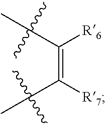
R′₁, R′₂, and R′₃ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂—NHR*, or adjacent two moieties combine to form a fused ring which may optionally contain 1-3 heteroatoms selected from halogen, 0, N, and S and which may be further substituted by one or more R*,
R′₄ and R′₅ are independently selected from —OH and —CO₂H;
R′₆ and R′₇ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂—NHR*, or R₆ and R₇ combine to form a fused ring, and
R* is independently selected at each occurrence from hydrogen or C₁-C₁₂ hydrocarbons each of which optionally contains 1-8 heteroatoms selected from halogen, O, N, and S and combinations thereof.

41. The method of claim 40, wherein when Q is 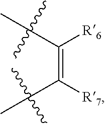
at least one of R′₁, R′₂, and R′₃ is not H.

42. The method of claim 40, wherein the additional compound has the structure of Formula (I′A): 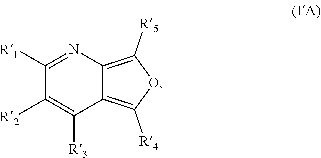
or a pharmaceutically acceptable salt thereof.

43. The method of claim 40, wherein the additional compound has the structure of Formula (I′B): 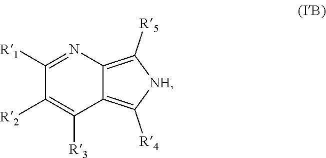
or a pharmaceutically acceptable salt thereof.

44. The method of claim 40, wherein the additional compound has the structure of Formula (I′C): 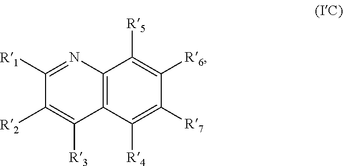
or a pharmaceutically acceptable salt thereof.

45. The method of claim 40, wherein the additional compound has the structure of Formula (I′D): 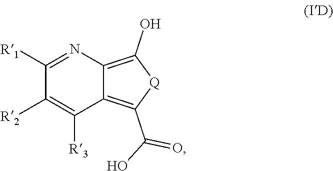
or a pharmaceutically acceptable salt thereof, wherein Q is —O— or —NH—.

46. The method of claim 40, wherein the additional compound has the structure of Formula (I′E): 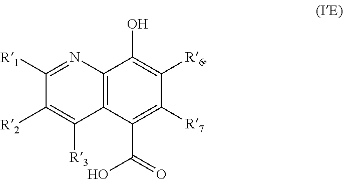
or a pharmaceutically acceptable salt thereof.

47. The method of claim 40, wherein the additional compound having the structure of Formula (I′) is selected from the group consisting of: 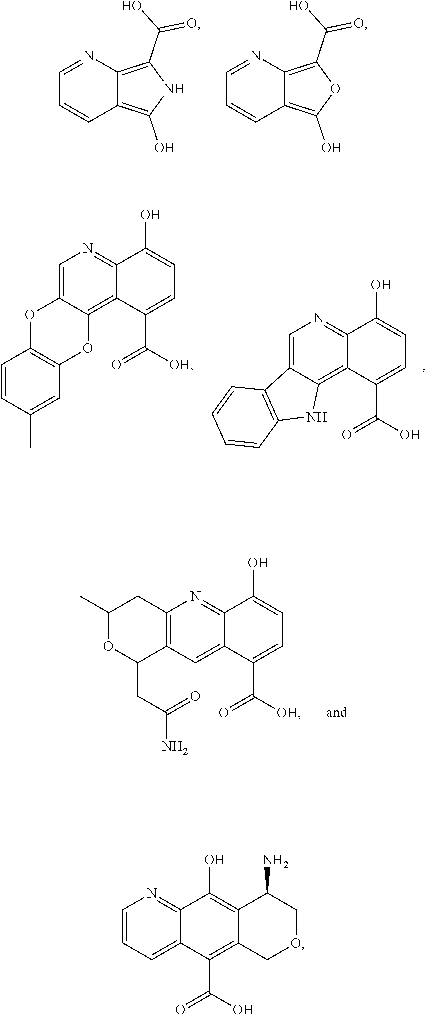 [SMILES: O=C(O)c1[nH]c(O)c2cccnc12 ; O=C(O)c1oc(O)c2cccnc12 ; Cc1ccc2c(c1)Oc1c(cnc3c(O)ccc(C(=O)O)c13)O2 ; O=C(O)c1ccc(O)c2ncc3c4ccccc4[nH]c3c12 ; CC1Cc2nc3c(O)ccc(C(=O)O)c3cc2C(CC(N)=O)O1 ; N[C@H]1COCc2c1c(O)c1ncccc1c2C(=O)O]
or a pharmaceutically acceptable salt thereof.

48. The method of claim 37, further comprising administering an additional compound having the structure of Formula (II′): 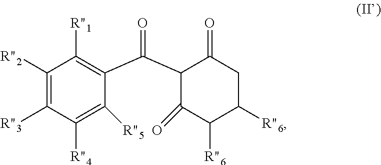
or a pharmaceutically acceptable salt thereof, wherein:
R″₁ is selected from —NO₂, —Cl, and a C₁₋₁₂ alkyl which may be optionally substituted with one or more of —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, and —SO₂—NHR*;
R″₂, R″₃, R″₄, R″₅, R″₆ and R″₇ are independently selected from hydrogen, C₁₋₁₂ alkyl, C₁₋₁₂ alkenyl, C₆₋₁₂ aryl, C₁₋₁₂ aralkyl, C₁₋₄ haloalkyl, or a combination thereof, each of which optionally contains 1-8 heteroatoms selected from halogen, O, N, and S; —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, —SO₂R*, —SO₂—NHR*, or adjacent two moieties combine to form a fused ring which may optionally contain 1-3 heteroatoms selected from halogen, O, N, and S and which may be further substituted by one or more R*, and
R* is independently selected at each occurrence from hydrogen or C₁-C₁₂ hydrocarbons each of which optionally contains 1-8 heteroatoms selected from halogen, O, N, and S and combinations thereof.

49. The method of claim 48, wherein the additional compound does not have the structure selected from

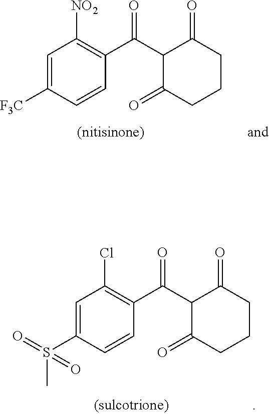 [SMILES: O=C1CCCC(=O)C1C(=O)c1ccc(C(F)(F)F)cc1[N+](=O)[O-] ; CS(=O)(=O)c1ccc(C(=O)C2C(=O)CCCC2=O)c(Cl)c1]

50. The method of claim 48, wherein the additional compound has the structure of Formula (II′A): 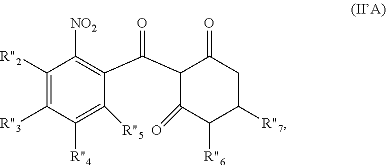
or a pharmaceutically acceptable salt thereof.

51. The method of claim 48, wherein the additional compound has the structure of Formula (II′B): 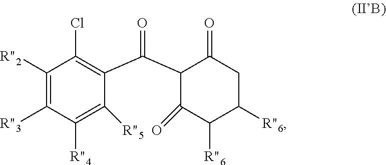
or a pharmaceutically acceptable salt thereof.

52. The method of claim 48, wherein R″₁ is a C₁₋₁₂ alkyl substituted with one or more of —OH, ═O, —CO₂H, —NO₂, —NH₂, —NHR*, —NR*₂, —N—OH, —HSO₃, —H₂PO₃, —OR*, —(C═O)—R*, —CO₂R*, —CO—NH₂, —CO—NHR*, and —SO₂—NHR*.

53. The method of claim 48, wherein the additional compound having the structure of Formula (II) is selected from the group consisting of: 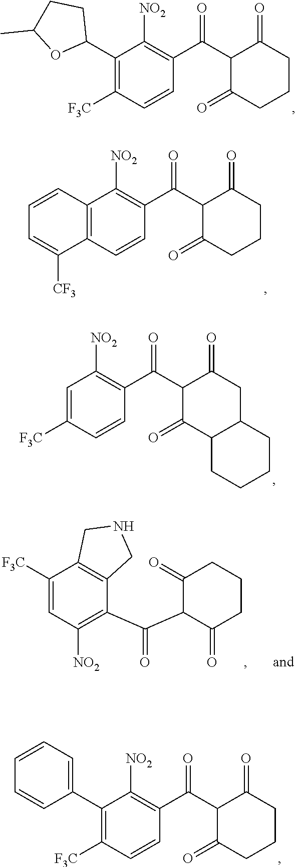 [SMILES: CC1CCC(c2c(C(F)(F)F)ccc(C(=O)C3C(=O)CCCC3=O)c2[N+](=O)[O-])O1 ; O=C1CCCC(=O)C1C(=O)c1ccc2c(C(F)(F)F)cccc2c1[N+](=O)[O-] ; O=C1CC2CCCCC2C(=O)C1C(=O)c1ccc(C(F)(F)F)cc1[N+](=O)[O-] ; O=C1CCCC(=O)C1C(=O)c1c([N+](=O)[O-])cc(C(F)(F)F)c2c1CNC2 ; O=C1CCCC(=O)C1C(=O)c1ccc(C(F)(F)F)c(-c2ccccc2)c1[N+](=O)[O-]]
or a pharmaceutically acceptable salt thereof.

54. The method of claim 48, wherein the additional compound having the structure of Formula (II′) is selected from the group consisting of: 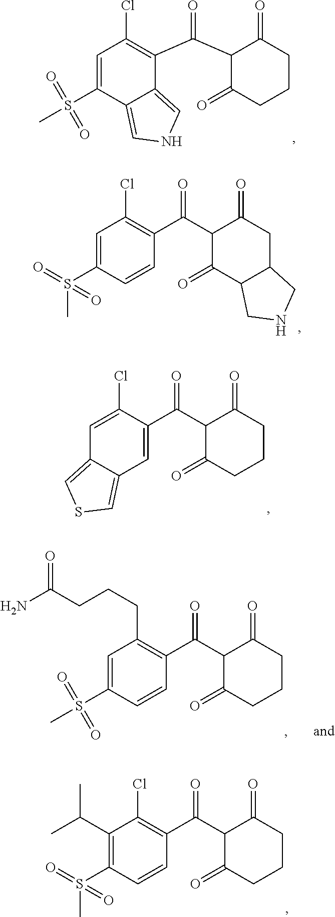
or a pharmaceutically acceptable salt thereof.

55. The method of claim 37, further comprising administering N-[5-[[4-[5-[acetyl(hydroxy)amino]pentylamino]-4-oxobutanoyl]-hydroxyamino]pentyl]-N-(5-aminopentyl)-N-hydroxybutanediamide (Deferoxamine; DFO).

56. The method of claim 37, wherein the single-agent chemotherapy is platinum-based chemotherapy.

57. The method of claim 56, wherein the method further comprises administering an electron transport chain (ETC) inhibitor in combination with the single-agent chemotherapy.

58. The method of claim 57, wherein the electron transport chain (ETC) inhibitor is metformin, phenformin, BAY84-2243, carboxyamidotriazole, ME344, Fenofibrate, mIBG (meta-iodobenzylguanidine), Alpha-TOS, Lonidamine, Atovaquone, Arsenic trioxide, Nitric Oxide, or Hydrocortisone, or a combination thereof.

59. The method of any one of claims 37-39, further comprising administering to the subject a multiagent chemotherapy, chemoradiotherapy, and/or neoadjuvant chemoradiotherapy if the subject is determined to have good prognosis.

60. The method of claim 59, wherein the multiagent chemotherapy is FOLFIRINOX (Leucovorin calcium/Folinic acid, 5-fluorouracil, Irinotecan, Oxaliplatin).

61. The method of any one of claims 1-60, wherein the HPDL expression level is determined at mRNA level via RNA-seq, reverse transcription polymerase chain reaction (rt-PCR), or fluorescence in situ hybridization (FISH).

62. The method of any one of claims 1-60, wherein the HPDL expression level is determined at protein level via immunohistochemistry (IHC) staining or immunofluorescence.

63. The method of claim 62, wherein the HPDL protein expression level is determined via chromogenic IHC staining using an anti-HPDL antibody as the primary antibody and an HRP-linked secondary antibody followed by DAB staining.

64. The method of any one of claims 1-63, wherein the cancer is pancreatic cancer.

65. The method of any one of claims 1-64, wherein the pancreatic cancer is pancreatic ductal adenocarcinoma (PDAC) or neuroendocrine pancreatic cancer.

66. The method of any one of claims 1-65, wherein the subject is a mammal.

67. The method of claim 66, wherein the subject is a human or an infant.
